SEQUENCE LISTING (6 sequences)

SEQ ID NO: 1 - DNA, 2787 bp, Homo sapiens
  source 1..2787: mol_type = genomic DNA
  misc_feature 1..2787: note = Gene Name: RB1 (RB transcriptional corepressor 1); note = Protein ID: NP_000312.2; note = Gene ID: 5925; note = CCDS: CCDS31973.1
atgccgcccaaaaccccccgaaaaacggccgccaccgccgccgctgccgccgcggaacccccggcaccgccgccgccgccccctcctgaggaggacccagagcaggacagcggcccggaggacctgcctctcgtcaggcttgagtttgaagaaacagaagaacctgattttactgcattatgtcagaaattaaagataccagatcatgtcagagagagagcttggttaacttgggagaaagtttcatctgtggatggagtattgggaggttatattcaaaagaaaaaggaactgtggggaatctgtatctttattgcagcagttgacctagatgagatgtcgttcacttttactgagctacagaaaaacatagaaatcagtgtccataaattctttaacttactaaaagaaattgataccagtaccaaagttgataatgctatgtcaagactgttgaagaagtatgatgtattgtttgcactcttcagcaaattggaaaggacatgtgaacttatatatttgacacaacccagcagttcgatatctactgaaataaattctgcattggtgctaaaagtttcttggatcacatttttattagctaaaggggaagtattacaaatggaagatgatctggtgatttcatttcagttaatgctatgtgtccttgactattttattaaactctcacctcccatgttgctcaaagaaccatataaaacagctgttatacccattaatggttcacctcgaacacccaggcgaggtcagaacaggagtgcacggatagcaaaacaactagaaaatgatacaagaattattgaagttctctgtaaagaacatgaatgtaatatagatgaggtgaaaaatgtttatttcaaaaattttataccttttatgaattctcttggacttgtaacatctaatggacttccagaggttgaaaatctttctaaacgatacgaagaaatttatcttaaaaataaagatctagatgcaagattatttttggatcatgataaaactcttcagactgattctatagacagttttgaaacacagagaacaccacgaaaaagtaaccttgatgaagaggtgaatgtaattcctccacacactccagttaggactgttatgaacactatccaacaattaatgatgattttaaattcagcaagtgatcaaccttcagaaaatctgatttcctattttaacaactgcacagtgaatccaaaagaaagtatactgaaaagagtgaaggatataggatacatctttaaagagaaatttgctaaagctgtgggacagggttgtgtcgaaattggatcacagcgatacaaacttggagttcgcttgtattaccgagtaatggaatccatgcttaaatcagaagaagaacgattatccattcaaaattttagcaaacttctgaatgacaacatttttcatatgtctttattggcgtgcgctcttgaggttgtaatggccacatatagcagaagtacatctcagaatcttgattctggaacagatttgtctttcccatggattctgaatgtgcttaatttaaaagcctttgatttttacaaagtgatcgaaagttttatcaaagcagaaggcaacttgacaagagaaatgataaaacatttagaacgatgtgaacatcgaatcatggaatcccttgcatggctctcagattcacctttatttgatcttattaaacaatcaaaggaccgagaaggaccaactgatcaccttgaatctgcttgtcctcttaatcttcctctccagaataatcacactgcagcagatatgtatctttctcctgtaagatctccaaagaaaaaaggttcaactacgcgtgtaaattctactgcaaatgcagagacacaagcaacctcagccttccagacccagaagccattgaaatctacctctctttcactgttttataaaaaagtgtatcggctagcctatctccggctaaatacactttgtgaacgccttctgtctgagcacccagaattagaacatatcatctggacccttttccagcacaccctgcagaatgagtatgaactcatgagagacaggcatttggaccaaattatgatgtgttccatgtatggcatatgcaaagtgaagaatatagaccttaaattcaaaatcattgtaacagcatacaaggatcttcctcatgctgttcaggagacattcaaacgtgttttgatcaaagaagaggagtatgattctattatagtattctataactcggtcttcatgcagagactgaaaacaaatattttgcagtatgcttccaccaggccccctaccttgtcaccaatacctcacattcctcgaagcccttacaagtttcctagttcacccttacggattcctggagggaacatctatatttcacccctgaagagtccatataaaatttcagaaggtctgccaacaccaacaaaaatgactccaagatcaagaatcttagtatcaattggtgaatcattcgggacttctgagaagttccagaaaataaatcagatggtatgtaacagcgaccgtgtgctcaaaagaagtgctgaaggaagcaaccctcctaaaccactgaaaaaactacgctttgatattgaaggatcagatgaagcagatggaagtaaacatctcccaggagagtccaaatttcagcagaaactggcagaaatgacttctactcgaacacgaatgcaaaagcagaaaatgaatgatagcatggatacctcaaacaaggaagagaaatga

SEQ ID NO: 2 - protein, 928 aa, Homo sapiens
MPPKTPRKTAATAAAAAAEPPAPPPPPPPEEDPEQDSGPEDLPLVRLEFEETEEPDFTALCQKLKIPDHVRERAWLTWEKVSSVDGVLGGYIQKKKELWGICIFIAAVDLDEMSFTFTELQKNIEISVHKFFNLLKEIDTSTKVDNAMSRLLKKYDVLFALFSKLERTCELIYLTQPSSSISTEINSALVLKVSWITFLLAKGEVLQMEDDLVISFQLMLCVLDYFIKLSPPMLLKEPYKTAVIPINGSPRTPRRGQNRSARIAKQLENDTRIIEVLCKEHECNIDEVKNVYFKNFIPFMNSLGLVTSNGLPEVENLSKRYEEIYLKNKDLDARLFLDHDKTLQTDSIDSFETQRTPRKSNLDEEVNVIPPHTPVRTVMNTIQQLMMILNSASDQPSENLISYFNNCTVNPKESILKRVKDIGYIFKEKFAKAVGQGCVEIGSQRYKLGVRLYYRVMESMLKSEEERLSIQNFSKLLNDNIFHMSLLACALEVVMATYSRSTSQNLDSGTDLSFPWILNVLNLKAFDFYKVIESFIKAEGNLTREMIKHLERCEHRIMESLAWLSDSPLFDLIKQSKDREGPTDHLESACPLNLPLQNNHTAADMYLSPVRSPKKKGSTTRVNSTANAETQATSAFQTQKPLKSTSLSLFYKKVYRLAYLRLNTLCERLLSEHPELEHIIWTLFQHTLQNEYELMRDRHLDQIMMCSMYGICKVKNIDLKFKIIVTAYKDLPHAVQETFKRVLIKEEEYDSIIVFYNSVFMQRLKTNILQYASTRPPTLSPIPHIPRSPYKFPSSPLRIPGGNIYISPLKSPYKISEGLPTPTKMTPRSRILVSIGESFGTSEKFQKINQMVCNSDRVLKRSAEGSNPPKPLKKLRFDIEGSDEADGSKHLPGESKFQQKLAEMTSTRTRMQKQKMNDSMDTSNKEEK

SEQ ID NO: 3 - DNA, 1314 bp, Homo sapiens
  source 1..1314: mol_type = genomic DNA
  misc_feature 1..1314: note = Gene ID: 1869; note = Protein ID: NP_005216-1; note = CCDS: CCDS13224.1; note = Gene Name: E2F1 (E2F transcription factor 1)
atggccttggccggggcccctgcgggcggcccatgcgcgccggcgctggaggccctgctcggggccggcgcgctgcggctgctcgactcctcgcagatcgtcatcatctccgccgcgcaggacgccagcgccccgccggctcccaccggccccgcggcgcccgccgccggcccctgcgaccctgacctgctgctcttcgccacaccgcaggcgccccggcccacacccagtgcgccgcggcccgcgctcggccgcccgccggtgaagcggaggctggacctggaaactgaccatcagtacctggccgagagcagtgggccagctcggggcagaggccgccatccaggaaaaggtgtgaaatccccgggggagaagtcacgctatgagacctcactgaatctgaccaccaagcgcttcctggagctgctgagccactcggctgacggtgtcgtcgacctgaactgggctgccgaggtgctgaaggtgcagaagcggcgcatctatgacatcaccaacgtccttgagggcatccagctcattgccaagaagtccaagaaccacatccagtggctgggcagccacaccacagtgggcgtcggcggacggcttgaggggttgacccaggacctccgacagctgcaggagagcgagcagcagctggaccacctgatgaatatctgtactacgcagctgcgcctgctctccgaggacactgacagccagcgcctggcctacgtgacgtgtcaggaccttcgtagcattgcagaccctgcagagcagatggttatggtgatcaaagcccctcctgagacccagctccaagccgtggactcttcggagaactttcagatctcccttaagagcaaacaaggcccgatcgatgttttcctgtgccctgaggagaccgtaggtgggatcagccctgggaagaccccatcccaggaggtcacttctgaggaggagaacagggccactgactctgccaccatagtgtcaccaccaccatcatctcccccctcatccctcaccacagatcccagccagtctctactcagcctggagcaagaaccgctgttgtcccggatgggcagcctgcgggctcccgtggacgaggaccgcctgtccccgctggtggcggccgactcgctcctggagcatgtgcgggaggacttctccggcctcctccctgaggagttcatcagcctttccccaccccacgaggccctcgactaccacttcggcctcgaggagggcgagggcatcagagacctcttcgactgtgactttggggacctcacccccctggatttctga

SEQ ID NO: 4 - protein, 437 aa, Homo sapiens
MALAGAPAGGPCAPALEALLGAGALRLLDSSQIVIISAAQDASAPPAPTGPAAPAAGPCDPDLLLFATPQAPRPTPSAPRPALGRPPVKRRLDLETDHQYLAESSGPARGRGRHPGKGVKSPGEKSRYETSLNLTTKRFLELLSHSADGVVDLNWAAEVLKVQKRRIYDITNVLEGIQLIAKKSKNHIQWLGSHTTVGVGGRLEGLTQDLRQLQESEQQLDHLMNICTTQLRLLSEDTDSQRLAYVTCQDLRSIADPAEQMVMVIKAPPETQLQAVDSSENFQISLKSKQGPIDVFLCPEETVGGISPGKTPSQEVTSEEENRATDSATIVSPPPSSPPSSLTTDPSQSLLSLEQEPLLSRMGSLRAPVDEDRLSPLVAADSLLEHVREDFSGLLPEEFISLSPPHEALDYHFGLEEGEGIRDLFDCDFGDLTPLDF

SEQ ID NO: 5 - RNA, 19 nt, synthetic construct
  source 1..19: mol_type = other RNA
  misc_feature 1..19: note = PRMT5 siRNA sequence
ccgctattgcaccttggaa

SEQ ID NO: 6 - RNA, 21 nt, synthetic construct
  source 1..21: mol_type = other RNA
  misc_feature 1..21: note = E2F-1 siRNA sequence
aactcctcgcagatcgtcatc

(19) United States
(12) Patent Application Publication
(10) Pub. No.: US 2026/0318918 A1
(43) Pub. Date: Oct. 15, 2026

(54) CANCER DIAGNOSTICS AND TREATMENT BY MEANS OF PRMT5 INHIBITOR

(71) Applicant: ARGONAUT THERAPEUTICS LIMITED, Oxford (GB)

(72) Inventors: Nicholas LA THANGUE, Oxford (GB); Shonagh MUNRO, Oxford (GB)

(21) Appl. No.: 18/846,797

(22) PCT Filed: Mar. 14, 2023
(86) PCT No.: PCT/GB2023/050601

§ 371 Date: Sep. 13, 2024

(30) Foreign Application Priority Data
Mar. 15, 2022 (GB) 2203588.5

(51) Int. Cl. G01N 33/575; C12Q 1/6886

(52) U.S. Cl. CPC G01N 33/575; C12Q 1/6886; C12Q 2600/106; C12Q 2600/158; G01N 2800/52

(57) ABSTRACT

The present invention relates to patient selection methods and methods of treating cancers with a protein arginine methyltransferase 5 (PRMT5) inhibitor. The expression level of the retinoblastoma tumour suppressor protein (pRb) and/or total E2F1 protein in a cancer cell containing biological sample from the patient is used to determine whether a patient should be treated with a PRMT5 inhibitor.

### FIELD OF THE INVENTION

The invention relates to the field of cancer. In particular, to patient selection methods and methods of treating cancers with a PRMT5 inhibitor. The present invention also provides kits for use in the methods of the invention.

### BACKGROUND TO THE INVENTION

The retinoblastoma protein (pRb)-E2F pathway is a key point of control in the cell cycle. It is often deregulated in tumour cells, and deregulation of the pathway is widely regarded as a ‘hallmark’ of cancer. Classically, the pRb tumour suppressor protein is a negative regulator of E2F transcription factors, which act as a transcriptional hub through which pRb exerts effects on cell cycle progression.

PRMT5 has been identified as a therapeutic target for treatment of cancer. Indeed, numerous patent publications disclose PRMT5 inhibitors and their use in treating cancer.

However, cancer is a heterogenous disease and amongst patients with a particular type of cancer (e.g. breast cancer or lung cancer) there are sub-populations of cancers. It is widely accepted that no drug works in the treatment of all persons with a particular type of cancer but the advent of personalised medicine, where it is possible to identify those patients most likely to respond well to a particular type of drug, for example using a diagnostic test for a gene mutation characteristic of response, has meant that likely responders to a drug can be identified and treated and likely non-responders excluded from treatment with a drug which will likely not work, but will probably cause toxic side-effects. These likely non-responders can thus be saved from inappropriate treatments and treated in some other fashion.

There is an ongoing need, particularly in the oncology field, for means to identify patients that will respond, or are most likely to respond, to a particular type of treatment.

E2F is a family of transcription factors implicated in a variety of cell fates including proliferation, apoptosis and differentiation (Stevens and La Thangue; 2003; Frolov and Dyson 2004, Polager and Ginsberg 2008; van den Heuvel and Dyson 2008). E2F proteins share the capacity to regulate a diverse group of target genes (Frolov and Dyson 2004; van den Heuvel and Dyson 2008). The first family member identified, E2F-1, physically interacts with the retinoblastoma tumour suppressor protein pRb, which negatively regulates E2F-1 activity (Bandara and La Thangue 1991; Zamanian and La Thangue 1992; Weinberg 1995; Stevens and La Thangue 2003). Whilst it is established that E2F-1 can promote proliferation, it has also become clear that E2F-1 can prompt apoptosis (van den Heuvel and Dyson 2008, Polager and Ginsberg 2008). In Rb−/− mice, the enhanced levels of apoptosis in certain tissues reflect deregulated E2F-1 activity (Tsai et el 1998; laquinta and Lees 2007). Further, E2F-1−/− mice suffer from an increased incidence of tumours (Field et al 1996), suggesting that E2F-1 adopts a tumour suppressor role in some tissues, perhaps reflecting its ability to induce apoptosis. However, the mechanisms that influence the diverse cellular outcomes that have been ascribed to E2F-1 activity, particularly its apoptotic activity and the cell context dependency of these events, remain elusive. It is an object of the invention to identify such mechanisms.

Not only is E2F-1 regulated during cell cycle progression (Stevens and La Thangue, 2003, van den Heuvel and Dyson 2008), but also under conditions of DNA damage (Pediconi et al 2003; Stevens et al 2003; Stevens and La Thangue 2003). In DNA damaged cells, E2F-1 is induced in a fashion that follows similar kinetics to p53 (Pediconi et al 2003; Stevens and La Thangue 2003), which co-incides with activation of a diverse collection of E2F target genes (Ren et al 2002). DNA damage activates a signal transduction pathway involving protein phosphokinases, such as ATM/ATR and Chkl/Chk2, which in turn phosphorylate effector proteins that mediate the outcome of the DNA damage response (Jackson and Bartek, 2009). Both families of DNA damage responsive kinases phosphorylate E2F-1, which contributes to the regulation of E2F-1 in DNA damaged cells (Stevens et al 2003; Stevens and La Thangue 2003). Moreover, E2F-1 prompts apoptosis under DNA damage conditions and, in tumour cells which harbour compromised p53 activity, might provide an important pathway that enables apoptosis to be activated (Stevens and La Thangue 2003).

The retinoblastoma tumour suppressor protein (pRb) is a key regulator of the G1 to S phase transition during the cell cycle, a process that is fundamental for correctly controlled cell proliferation (Dick and Rubin, 2013). pRb/Rb1 loss is common amongst all cancer types and this is strongly associated with poor overall survival (Knudsen et al, 2020 and Ertel et al, 2010). Loss of Rb in cancer can occur by deletion of one or both copies of the gene, mutations resulting in non-functional protein or by promoter methylation of the Rb gene (Hanahan and Weinberg, 2000). In addition to mutation of the RB1 gene itself, upstream Rb pathway aberrations can occur in a high proportion of human cancers, for example inactivation of CDK inhibitors and activation of cyclin and CDK are frequently occurring events (Hanahan and Weinberg, 2000).

PRMT5 is a member of the protein arginine methyltransferase (PRMT) family. PRMT5 is a type II methyltransferase that catalyses the symmetric dimethylation of its substrate proteins (Bedford and Clarke et al, 2009). Arginine methylation is known to play a role in a wide variety of cellular processes. In recent years there have been an increasing number of reports that have highlighted the potential role of PRMT5 as a driver of oncogenesis (Jarrold and Davis, 2019, Kim and Ronai, 2020). PRMT5 has been shown to promote tumorigenesis in a wide range of cancers including haematological malignancies, colon, breast, prostate, lung, liver, bone, skin, ovarian, gastric, brain, and pancreatic cancers (Shailesh et al, 2018).

E2F-1 has been shown to be a target for methylation by the protein arginine methyltransferase, PRMT5. Methylation of E2F1 by PRMT5 promotes cell growth by increasing the transcription of cell cycle related E2F1 target genes (Cho et al, 2012 and Zheng et al, 2013). Furthermore, subsets of genes under control by E2F1 and PRMT5 have been identified that can influence cancer cell viability, migration, invasion and adherence (Barczak et al, 2020). There is a common coincidence of high levels of expression of both E2F1 and PRMT5 in a number of cancers and this has been found to correlated with poor prognosis (Barczak et al, 2020).

There is a need in the art for methods that can select the patients that are most likely to respond favourably to cancer treatment with a PRMT5 inhibitor. The present invention addresses that need.

### SUMMARY OF THE INVENTION

According to a first aspect of the invention there is provided a method of selecting a treatment for a patient with cancer comprising:
(i) determining the expression level of biomarker (a) pRb and/or (b) total E2F1 in a cancer cell containing biological sample from the patient;
(ii) comparing the expression level(s) in (i) with a reference value for each biomarker,
(iii) wherein if the patient's cancer cells exhibit a decrease in expression of pRb and/or an increase in expression of total E2F1 compared to the reference value the patient is selected for treatment with a PRMT5 inhibitor.

The expression level of pRb can be determined by quantifying the amount of pRb protein in the sample or the amount of RB1 transcript. The expression level of total E2F1 can be determined by quantifying the amount of total E2F1 protein in the sample or the amount of E2F1 transcript.

Total E2F-1 refers to the totality of all types of E2F-1, rather than just one form of E2F-1 such as methylated E2-F1.

According to a second aspect of the invention there is provided a method of selecting a treatment for a patient with cancer comprising determining whether the PRB1 gene in the cancer cells comprises one or more mutations that result in reduced, including null, expression of pRb, wherein if the cancer cells comprise one or more mutations in RB1 gene that result in reduced expression of pRb the patient is selected for treatment with a PRMT5 inhibitor.

According to a third aspect of the invention there is provided a kit for use in the method of the first aspect of the invention, which kit comprises one or more reagents capable of determining the expression level of pRb and/or total E2F1. Suitably, the kit comprises an antibody or antigen-binding portion thereof which specifically binds to E2F-1 protein; and/or an antibody or antigen-binding portion thereof which specifically binds to pRb protein; and/or a nucleic acid oligonucleotide capable of specifically binding to RB1 transcript; and/or a nucleic acid oligonucleotide capable of specifically binding to E2F1 transcript.

According to a fourth aspect of the invention there is provided a PRMT5 inhibitor for use in treating a patient identified according to the first aspect of the invention.

In particular, according to a variation of the fourth aspect of the invention there is provided a PRMT5 inhibitor for use in treating a cancer whose cells express greater than normal levels of total E2F1 protein and/or reduced levels of pRb protein compared to normal.

According to a fifth aspect of the invention there is provided a PRMT5 inhibitor for use in treating a pRb defective cancer or for use in treating a cancer whose cells express reduced levels of pRb biomarker compared to normal.

Suitably a pRb defective cancer is one that comprises cancer cells that comprise one or more mutations in RB1 gene that result in reduced, including null, expression of pRb in the cancer cell.

Any agent capable of inhibiting PRMT5 can be utilised in the fourth or fifth aspects of the invention. Suitably, the PRMT5 inhibitor is a small molecule compound, an antisense oligonucleotide, RNAi molecule or an antibody or binding-fragment thereof.

According to a sixth aspect of the invention there is provided a computer-implemented method to aid in selecting a treatment for a patient with cancer, comprising the steps of:
(i) receiving a value for the level of biomarker (a) pRb and/or (b) total E2F1 in a cancer cell containing biological sample from the patient;
(ii) comparing the level(s) in (i) with a reference value for each biomarker,
(iii) wherein if the patient's cancer cells exhibit a decrease in level of pRb and/or an increase in level of total E2F1 compared to the reference value the patient is selected for treatment with a PRMT5 inhibitor.

Suitably, the level is expression level.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Other features and advantages of the invention will be apparent from the following:

### DETAILED DESCRIPTION OF THE INVENTION

The disclosed methods may be understood more readily by reference to the following detailed description which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

The methods of the present disclosure will employ, unless otherwise indicated, conventional techniques of molecular biology (including recombinant techniques), microbiology, cell biology, and biochemistry, which are within the skill of the art. Exemplary techniques are explained fully in the literature, such as, “Molecular Cloning: A Laboratory Manual”, 2^{nd} edition (Sambrook et al., Molecular Cloning: A Laboratory Manual, New York: Cold Spring Harbor Press, 1989); “Current Protocols in Molecular Biology” (F. M. Ausubel et al., eds., Current Protocols of Molecular Biology, John Wiley and Sons (1987); and “PCR: The Polymerase Chain Reaction”, (Mullis et al., eds., Birhauser, Boston, 1994).

In describing the present invention, the following terms will be employed, and are intended to be defined as indicated below.

It is to be appreciated that certain features of the disclosed methods, which are for clarity, described herein in the context of separate embodiments, may also be provided in combination in a single embodiment. Conversely, various features of the disclosed methods that are, for brevity, described in the context of a single embodiments, may also be provided separately or in any sub-combination.

The articles “a,” “an,” and “the” are used herein to refer to one or to more than one (i.e. to at least one) of the grammatical object of the article.

The use of the alternative (e.g., “or”) should be understood to mean either one, both, or any combination thereof of the alternatives.

The term “and/or” should be understood to mean either one, or both of the alternatives.

As used herein and unless stated otherwise, it is to be understood that the term “about” is used synonymously with the term “approximately”. Illustratively and unless stated otherwise, the use of the term “about” when used in conjunction with a stated numerical value or range denotes somewhat more or somewhat less than the stated value or range, to within a range of ±15% of that stated, ±10% of that stated, ±5% of that stated, or conveniently ±2% of that stated. Such values are thus encompassed by the scope of the claims reciting the terms “about” or “approximately”.

As used herein, “cancer sample” or “cancer cell containing sample” means any biological sample containing one or more cancer cells, or one or more cancer derived RNAs or proteins, and obtained from a cancer patient. For example, a tissue sample obtained from a cancer tissue of a cancer patient is a useful cancer sample in the present invention. The tissue sample can be a formalin fixed, paraffin embedded (FFPE) sample, or fresh frozen sample, and preferably contain largely cancer cells.

As used herein, the term “effective amount” or “therapeutically effective amount” refers to an amount of an agent which confers a therapeutic effect on a treated subject, at a reasonable benefit/risk ratio applicable to any medical treatment. A therapeutic effect may be objective (i.e., measurable by some test or marker) or subjective (i.e., subject gives an indication of or feels an effect). In particular, an “effective amount” refers to an amount of a therapeutic agent effective to treat, ameliorate, or prevent a desired disease or condition, or to exhibit a detectable therapeutic or preventative effect, such as by ameliorating symptoms associated with a disease, preventing or delaying onset of a disease, and/or also lessening severity or frequency of symptoms of a disease.

An effective amount is commonly administered in a dosing regimen that may comprise multiple unit doses. For any particular therapeutic agent, an effective amount (and/or an appropriate unit dose within an effective dosing regimen) may vary, for example, depending on route of administration, on combination with other agents. Also, a specific effective amount (and/or unit dose) for any particular patient may depend upon a variety of factors including what disorder is being treated; disorder severity; activity of specific agents employed; specific composition employed; age, body weight, general health, and diet of a patient; time of administration, route of administration; treatment duration; and like factors as is well known in the medical arts. The therapeutically effective amount is typically the dosage of the agent as approved by a national health authority (such as the US Food and Drug Administration [FDA] or European Medicines Agency [EMA]) which will have been identified from controlled human clinical trials.

The term “inhibitor” as used herein, refers to an entity/agent whose presence in a system in which an activity of interest is observed correlates with a decrease in level and/or nature of that activity as compared with that observed under otherwise comparable conditions when the inhibitor is absent. In some embodiments, an inhibitor interacts directly with a target whose activity is of interest. In some embodiments, an inhibitor affects the amount/level of a target of interest; alternatively, or additionally, in some embodiments, an inhibitor affects the activity of a target of interest without affecting the level of the target. In some embodiments, an inhibitor affects both level and activity of a target entity of interest, so that an observed difference in activity is not entirely explained by or commensurate with an observed difference in level. The inhibitor can be any agent, e.g. small molecule compound, nucleic acid, antibody, and the like. The target can be a protein or a precursor thereof, or nucleic acid encoding said protein/precursor, e.g. genomic DNA or mRNA.

The term “oligonucleotide” refers to a relatively short polynucleotide, including, without limitation, single-stranded deoxyribonucleotides, single- or double-stranded ribonucleotides, RNA/DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

The term “polynucleotide,” when used in singular or plural, generally refers to any polyribonucleotide or polydeoxyribonucleotide, which may be unmodified RNA or DNA or modified RNA or DNA.

As used herein, the term “primer” refers to a molecule, typically a single-stranded oligonucleotide that can be used to generate a PCR reaction amplification product (amplicon). A primer is sometimes referred to as PCR primer.

As used herein, the term “probe” refers to a molecule, typically a single-stranded oligonucleotide that can be used to detect a complementary target nucleic acid product by hybridising thereto due to the sequence complementarity. The probe may be labelled, such as with a fluorescent marker or other label to facilitate detection. A probe is sometimes referred to as hybridisation primer.

The term “RNA transcript” as used herein refers to the RNA transcription products of a gene, including, for example, mRNA, an unspliced RNA, a splice variant mRNA, a microRNA, and a fragmented RNA.

As used herein, by “selectively hybridize” we mean capable of only hybridizing to a unique complementary single region of the target in a sample, e.g. under conditions which allow hybridization to the target nucleic acid selectively. In this way, each primer or probe can only hybridize to one target sequence, thus avoiding off target binding.

Selective hybridization typically occurs when two nucleic acid sequences are substantially complementary (at least about 75% complementary over a stretch of at least 14 to 25 nucleotides, preferably at least about 85% complementary, more preferably at least about 90% complementary). As a result, it is expected that a certain degree of mismatch is tolerated.

Those skilled in the art are able to employ suitable conditions of the desired stringency for selective hybridization, taking into account factors such as oligonucleotide length and base composition, temperature and so on.

Suitable selective hybridization conditions for oligonucleotides of approximately 17-35 bases include hybridization for an hour at 42° C. in 6×SSC and washing in 6×SSC at a series of increasing temperatures from 42° C. to 65° C. For example, the wash may be carried out using 6×SSC at 42° C. for 30 minutes, then 6×SSC at 50° C. for 45 minutes, then 2×SSC for 45 minutes at 65° C. When conducting a PCR reaction conditions for selective hybridization could involve reacting the target nucleic acid and probe/primer at about 50-60° C. for 5-10 minutes in a suitable buffer.

The degree of stringency of washing can be varied by changing the temperature, pH, ionic strength, divalent cation concentration, volume and duration of the washing. For example, the stringency of hybridization may be varied by conducting the hybridization at varying temperatures below the melting temperatures of the primer or probes. For oligonucleotide probes, between 14 and 70 nucleotides in length, the melting temperature (Tm) in degrees Celsius may be calculated using the formula: Tm=81.5+16.6(log [Na+])+0.41(fraction G+C)−(600/N) where N is the length of the oligonucleotide.

Other suitable conditions and protocols are described in Molecular Cloning: A Laboratory Manual; 4^{th} Edition, Green & Sambrook (2012) Cold Spring Harbor Laboratory Press NY; and, Current Protocols in Molecular Biology, Ausubel et al. eds. John Wiley & Sons (2003).

As used herein, “subject” includes a vertebrate, mammal, domestic animal or preferably a human being.

A “subject,” “individual,” or “patient” as used herein, includes any animal that exhibits a symptom of a condition that can be detected or identified with compositions contemplated herein. Suitable subjects include laboratory animals (such as mouse, rat, rabbit, or guinea pig), farm animals (such as horses, cows, sheep, pigs), and domestic animals or pets (such as a cat or dog). In particular embodiments, the subject is a mammal. In certain embodiments, the subject is a non-human primate and, in a particular embodiment, the subject is a human.

An individual who is “suffering from” a disease, disorder, and/or condition has been diagnosed with and/or displays one or more symptoms of the disease, disorder, and/or condition. When referred to herein, an individual who is suffering from a disease (e.g. cancer) is also one who has the disease (e.g. cancer) or one who is in need of treatment for the disease (e.g. cancer).

As used herein, the term “treatment” (also “treat” or “treating”) refers to any administration of a substance (e.g. PRMT5 inhibitor) that partially or completely alleviates, ameliorates, relives, inhibits, delays onset of, reduces severity of, and/or reduces frequency, incidence or severity of one or more symptoms, features, and/or causes of a particular disease, disorder, and/or condition (e.g. cancer). Such treatment may be of a subject who does not exhibit signs of the relevant disease, disorder and/or condition and/or of a subject who exhibits only early signs of the disease, disorder, and/or condition. Alternatively, or additionally, such treatment may be of a subject who exhibits one or more established signs of the relevant disease, disorder and/or condition. In some embodiments, treatment may be of a subject who has been diagnosed as suffering from the relevant disease, disorder, and/or condition. The treatment can be part of “a method of treatment” which may include the diagnosis or selection of the patient/individual as well as the therapeutic intervention. The selection of the patient may involve testing the patient for their suitability to be treated by the therapeutic intervention, which may involve testing to determine whether the patient's cancer has a relevant deficiency in a protein or encoding nucleic acid. In the context of the present invention this could be testing to see if the cancer comprises cells that express higher levels of total E2F1 than normal; and/or express lower levels of pRb than normal; and/or comprise one or more mutations in PRB1 gene that result in reduced, including null, expression of pRb in the cancer cell. Suitably, such testing is carried out in vitro on a biological sample comprising cancer cells from the subject with or suspected of having cancer.

### Diagnostic Methods

The present invention arises from the recognition that the cancer cell targeting effect of PRMT5 inhibitor was significantly increased in cancers that express higher than normal levels of E2F1, particularly those that have high levels of total E2F1 than normal; additionally, the cancer cell targeting effect of PRMT5 inhibitor was significantly increased in cancers that express lower than normal levels of pRb (including no expression). Thus, cancer cells that have the phenotype of enhanced total EF21 expression and/or reduced pRb expression are most suited for treatment with a PRMT5 inhibitor. This finding thus offers up the opportunity to select patients most suited to effective anti-cancer therapy with a PRMT5 inhibitor.

Thus, according to a first aspect of the invention there is provided a method of selecting a treatment for a patient with cancer comprising:
(i) determining the expression level of biomarker (a) pRb and/or (b) total E2F1 in a cancer cell containing biological sample from the patient;
(ii) comparing the expression level(s) in (i) with a reference value for each biomarker,
(iii) wherein if the patient's cancer cells exhibit a decrease in expression of pRb and/or an increase in expression of total E2F1 compared to the reference value the patient is selected for treatment with a PRMT5 inhibitor.

The expression level of a biomarker, such as pRb, can be determined by quantifying the amount of protein in the sample or the amount of transcript that encodes the protein. In humans, the retinoblastoma protein (protein name abbreviated pRb; gene name abbreviated Rb, RB or RB1) is encoded by the RB1 gene located on chromosome 13, at 13q14.1-q14.2. A reference sequence for the protein is disclosed in NCBI with the Reference sequence NP_000312. A reference sequence for the RB1 gene is disclosed in NCBI with the Reference sequence CCDS31973.1. Reference sequences are also available from G!Ensembl under ENSG00000139687 and UniProtKB under reference P06400.

The RB1 nucleotide sequence is disclosed in SEQ ID NO: 1.

The translated protein (pRb) sequence is disclosed in SEQ ID NO: 2.

In humans, the E2F transcription factor 1 (E2F1) is located on chromosome 20. The gene maps to 32,263,283-32,274,191 in GRCh37 coordinates.

A reference sequence for the protein is disclosed in NCBI with the Reference sequence NP_005216-1. A reference sequence for the E2F1 gene is disclosed in NCBI with the Reference sequence CCDS13224.1. Reference sequences are also available from G!Ensembl under ENSG00000101412.

The E2F1 nucleotide sequence is disclosed in SEQ ID NO: 3.

The translated protein sequence is disclosed in SEQ ID NO: 4.

These sequences can be used to design nucleic acid probes or primer capable of binding to the transcript, or antibodies to the proteins suitable for use in the invention disclosed herein.

A comparison of the expression level of a biomarker (e.g. amount of protein or transcript) in the cancer cell sample must be made to assess whether the level has gone up, down or stayed the same. Such comparison can be made to a reference value indicative of wild-type or normal biomarker levels. Such reference level can be determined by assaying a wild type cell containing biological sample in parallel with the cancer cell containing biological sample from the patient and comparing the expression level of the biomarker(s) in each sample. Suitably the two samples comprise the approximate same number and type of cells so as to ensure comparability. Thus, if the cancer cell sample is a breast tissue sample, the comparator sample could be a matched non-cancerous breast tissue sample from the same patient or a different subject. More appropriately, the typical expression levels of the biomarker in matched non-cancerous tissues will have been predetermined from analysis of numerous samples from non-cancerous tissues (e.g. from healthy subjects). In one approach an average of such wild-type levels can be used as the reference value. In another approach a threshold value can be taken from the wild-type levels (e.g. the 5 percentile value or 95 percentile value). Thus, for example, when seeking to detect an increase in expression level of a biomarker in a test sample, the value indicative of the value representing the 95% highest value may be taken as the reference value. Alternatively, when seeking to detect a decrease in expression level of a biomarker in a test sample, the value indicative of the value representing the 5% highest value may be taken as reference value. It will be appreciated that the 5% and 95% values used herein is merely illustrative. The actual reference value for each biomarker is likely to be established from appropriately controlled clinical studies.

Mutation in RB1 gene can lead to a reduced expression of pRb relative to normal levels (Derenzini et al, 2008, Benedict et al, 1999, Bhateja et al, 2019, Lacombe et al, 2021, Xing et al, 1999). Accordingly, a patient's cancer can be determined to be one that has a reduced expression level of pRb by detecting for one or more mutations in pRB gene that cause a reduced expression.

Thus, according to a second aspect of the invention there is provided a method of selecting a treatment for a patient with cancer comprising determining whether the RB1 gene in the cancer cells comprises one or more mutations that result in reduced, including null, expression of pRb, wherein if the cancer cells comprise one or more mutations in RB1 gene that result in reduced, including null, expression of pRb in the cancer cell the patient is selected for treatment with a PRMT5 inhibitor.

Suitably, the analysis is conducted in vitro (which includes ex vivo) on a cancer cell sample that has been previously isolated from the patient.

Suitably, the mutation is one selected from E137X, R251X, R255X, R320X, R358X, R445X, R455X, R467X, R552X, R556X, R579X, R787X, R661W, C712R.

The mutation identifier follows the established practise of identifying the amino acid in wild-type protein, the location of the amino acid and then the amino acid that is substituted in the mutant form, thus, C712R identifies arginine (R) substitution of the cysteine (C) at position 712. X refers to substitution to any amino acid.

The person skilled in the art can easily devise probes or primers capable of specifically identifying the presence of nucleic acids that encode the above amino acid substitution mutations.

The presence of a mutation in RB1 gene can be determined using any number of well-established techniques, including nucleic acid sequencing; or using an amplification technique (such as polymerase chain reaction) with one or more primers that be acted on to produce an extension product only when a particular base mutation is present (e.g. allele-specific amplification); or with one or more probes that can selectively discriminate between nucleic acids that have or do not have one or more mutations (e.g. allele-specific hybridisation). Preferably, the presence or absence of a mutation within RB1 gene is determined by nucleic acid sequencing.

### Sample

The levels of a biomarker (e.g. E2F1 or pRb), or the presence of one or more mutations in RB1 gene can be determined from any suitable cancer cell containing sample (cancer sample) of the patient.

The skilled person will appreciate that there are many suitable examples of cancer cell sample that can be employed. A suitable biological sample may be a tissue sample, such as a sample from a biopsy or surgical resection, or a biofluid sample that comprises tumour cells, such as blood, plasma, serum, sputum, saliva, pleural effusion, ascites, urine and the like. Suitably, the sample is fresh, frozen (e.g. fresh frozen), or paraffin-embedded and fixed (e.g. formalin-fixed).

In a particular embodiment, the cancer sample has been previously isolated from the patient, optionally as a solid or liquid biopsy sample or during surgery.

In one embodiment the sample isolation is part of the diagnostic method.

Suitably the cancer sample is isolated during surgical tumour resection, or from a solid or liquid biopsy, such as fine-needle aspiration biopsy, core needle biopsy or liquid biopsy (e.g. blood or ascites sample).

In particular embodiments, the cancer sample is fresh, frozen, or paraffin-embedded and fixed.

### Determining Protein Level

The expression level of each biomarker can be determined based on the amount of protein present in the sample.

In one embodiment of the invention, biomarker expression level may be determined at the protein level. Such methods are well known in the art and include, e.g., any immunohistochemistry (IHC) based, antibody (including autoantibodies against the protein) based, mass spectroscopy based, and image (including used of labelled ligand) based method known in the art and recognized as appropriate for the detection of proteins.

Normalisation against reference proteins can then be carried out to facilitate quantitation of biomarker. The normalised value can then be compared to wild-type/normal cell biomarker expression levels or a threshold value to see whether the cancer is one that expresses mor or less of a measured biomarker than wild-type, and/or to classify the cancer as one likely or not-likely to respond favourably to a PRMT5 inhibitor.

In a particular embodiment, the expression level for each biomarker (e.g. E2F1 or pRb) is determined based on the amount of the biomarker protein detected.

In one embodiment the detection is via an immunoassay that uses one or more antibodies specific for one or more epitopes of the biomarker protein in a cell sample of interest. Any biological material can be used for the detection/quantification of the biomarker protein.

The biomarker proteins can be detected in any suitable manner but are typically detected by contacting a sample from the patient (i.e. one containing cancer cells) with an antibody that binds the biomarker protein and then detecting the presence or absence of a reaction product. Such as, by use of labelled antibodies against cell surface markers followed by fluorescence activated cell sorting (FACS). Such antibodies are preferably labelled to permit their easy detection after binding to the gene product. Detection methodologies suitable for use in the practice of the invention include, but are not limited to, immunohistochemistry of cell containing samples or tissue, enzyme linked immunosorbent assays (ELISAs) including antibody sandwich assays of cell containing tissues or blood samples, mass spectroscopy, and immuno-PCR.

Antibodies that can be used herein may be monoclonal, polyclonal, chimeric, or a fragment of the foregoing. Suitably, the antibody is a monoclonal antibody. Suitably, the step of detecting the reaction product may be carried out with any suitable immunoassay.

Antibodies to known proteins, including labelled antibodies, are often commercially available. However, an antibody capable of specifically binding to one of the biomarkers (e.g. E2F1 or pRb) can be prepared using well-established protocols. General procedures for antibody preparation are described in Harlow, E. and Lane, D., Using Antibodies: A Laboratory Manual, Cold Spring Harbor Press, New York, 1999; Zola, H., Monoclonal Antibodies: A Manual of Techniques, CRC Press, Inc., Boca Raton, Florida, 1984; And Coligan, CURRENT PROTOCOLS IN IMMUNOLOGY, Wiley/Greene, NY, 1991, which are incorporated herein by reference.

As well as full-size/intact antibody, which can be of any immunoglobulin class including IgG, IgM, IgE, IgA, IgD and any subclass thereof, a fragment thereof (e.g., Fab or F(ab′)2), or an engineered variant thereof (e.g., sFv) can also be used. Such moieties are collectively referred to as antigen-binding moieties. An antigen binding moiety is optionally conjugated with a detectable label.

Techniques for detecting antibody binding through the use of a detectable label are well known in the art. For example, antibody binding may be detected through the use of chemical reagents that generate a detectable signal that corresponds to the level of antibody binding and, accordingly, to the level

of biomarker protein expression. In some embodiments, the detection antibody is coupled to an enzyme, particularly an enzyme that catalyses the deposition of a chromogen at the antigen-antibody binding site. Suitable enzymes include but are not limited to horseradish peroxidase (HRP) and alkaline phosphatase (AP). Commercial antibody detection systems may also be used to practice the invention. As used herein, antibody binding also cover binding by an antigen binding moiety.

Although antibodies/antigen binding moieties are illustrated herein for use in the invention because of their extensive characterization, any other suitable agent (e.g., a peptide, an aptamer, or a small organic molecule) that specifically binds a biomarker is optionally used in place of the antibody. For example, an aptamer that specifically binds a selected biomarker may be used. Aptamers are nucleic acid-based molecules that bind specific ligands. Methods for making aptamers with a particular binding specificity are known in the art.

As described elsewhere, the sample from the subject is typically a solid tissue sample, e.g. a biopsy, as described above, but may be a cancer cell containing biological fluid, e.g. blood or serum sample. The sample may be in the form of a tissue specimen from a patient where the specimen is suitable for immunohistochemistry in a variety of formats such as paraffin-embedded tissue, frozen sections of tissue, and freshly isolated tissue. The immunodetection methods are antibody-based but there are numerous additional techniques that allow for highly sensitive determinations of binding to an antibody in the context of a tissue. Those skilled in the art will be familiar with various immunohistochemistry strategies.

Immunoassays carried out in accordance with the present invention may be homogeneous assays or heterogeneous assays. In a homogeneous assay the immunological reaction usually involves the specific antibody (e.g., anti-biomarker protein antibody), a labelled analyte, and the sample of interest. The signal arising from the label is modified, directly or indirectly, upon the binding of the antibody to the labelled analyte. Both the immunological reaction and detection of the extent thereof are carried out in a homogeneous solution. Immunochemical labels that may be employed include free radicals, radioisotopes, fluorescent dyes, enzymes, bacteriophages, or coenzymes.

In a heterogeneous assay approach, the reagents are usually the sample, the antibody, and means for producing a detectable signal. Samples as described above may be used. The antibody is generally immobilized on a support, such as a bead, plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the sample. Means for producing a detectable signal include the use of radioactive labels, fluorescent labels, or enzyme labels.

For example, if the protein (or polypeptide) to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are radioimmunoassays, immunofluorescence methods, chemiluminescence methods, electrochemiluminescence or enzyme-linked immunoassays.

Those skilled in the art will be familiar with numerous specific immunoassay formats and variations thereof, which may be useful for carrying out the method disclosed herein. See generally E. Maggio, Enzyme-Immunoassay, (1980) (CRC Press, Inc., Boca Raton, Fla.); see also U.S. Pat. No. 4,727,022 to Skold et al. titled “Methods for Modulating Ligand-Receptor Interactions and their Application,” U.S. Pat. No. 4,659,678 to Forrest et al. titled “Immunoassay of Antigens,” U.S. Pat. No. 4,376,110 to David et al., titled “Immunometric Assays Using Monoclonal Antibodies,” U.S. Pat. No. 4,275,149 to Litman et al., titled “Macromolecular Environment Control in Specific Receptor Assays,” U.S. Pat. No. 4,233,402 to Maggio et al., titled “Reagents and Method Employing Channeling,” and U.S. Pat. No. 4,230,767 to Boguslaski et al., titled “Heterogenous Specific Binding Assay Employing a Coenzyme as Label.” Antibodies/antigen binding moieties may be conjugated to a solid support suitable for a diagnostic assay (e.g., beads, plates, slides or wells formed from materials such as latex or polystyrene) in accordance with known techniques, such as passive binding.

Antibodies/antigen binding moieties as described herein may likewise be conjugated to detectable groups such as radiolabels (e.g., 35S, 1251, 1311), enzyme labels (e.g., horseradish peroxidase, alkaline phosphatase), and fluorescent labels (e.g., fluorescein) in accordance with known techniques.

Alternative methods of detecting a protein biomarker in a sample include high performance liquid chromatography (HPLC) and other high-throughput techniques.

Additionally, identification and quantification of one or more biomarkers can be performed using mass spectrometry. One specific example of mass spectrometry that may be useful is tandem mass spectrometry; another example is high mass accuracy/high mass resolution mass spectrometry (e.g. Orbitrap™, Thermo Scientific).

Tandem mass spectrometry, for example, can be used for quantitative analysis of peptides in biological samples due to high sensitivity and specificity. High mass accuracy/high mass resolution mass spectrometers (e.g. Orbitrap™, Thermo Scientific) also can be utilized for analysis.

### Determining RNA Transcript Level

RNA transcript expression levels can be used as a surrogate measure of the level of protein in a sample. The RNA transcript expression level can be determined either at the RNA level (i.e., mRNA or noncoding RNA (ncRNA)) (e.g., miRNA, tRNA, rRNA, snoRNA, siRNA and piRNA) or at the protein level. Measuring gene expression at the mRNA level includes measuring levels of cDNA corresponding to mRNA.

Those skilled in the art are familiar with various techniques for determining the status of a gene or protein in a tissue or cell sample including, but not limited to, microarray analysis (e.g., for assaying mRNA or microRNA expression, copy number, etc.), real-time PCR (RTPCR), quantitative real-time PCR (qRT-PCR, e.g., TaqMan™), digital PCR (dPCR), microarrays, high-throughput sequencing (also known as next generation sequencing, e.g. RNA-seq), serial analysis of gene expression (SAGE) and Digital Gene Expression (DGE), etc.

In particular embodiments, the expression level of each biomarker gene may be determined in relation to various features of the expression products of the gene including exons, introns, protein epitopes and protein activity.

In a particular embodiment, the RNA transcript expression level for each measured gene is determined quantitatively. Quantitative measurements typically involve parallel measurement of the expression levels of one or more reference or housekeeping genes so as to determine the normalised expression level of the test RNA transcript. This is to ensure that approximately the same amount of test sample is being compared to the same amount of control sample and/or to the reference or threshold values.

In a particular embodiment, the determination of transcript expression level in accordance with the first or second aspects of the invention is performed using RT-PCR.

In a particular embodiment, the RT-PCR is quantitative reverse-transcription polymerase chain reaction (RT-qPCR).

Messenger RNA isolation, purification, primer extension and amplification can be performed according to methods available in the art. (see, e.g., Godfrey et al. J. Molec. Diagnostics 2: 84-91 (2000); Specht et al., Am. J. Pathol. 158: 419-29 (2001)). TaqMan® RT-PCR can be performed using commercially available equipment, such as, for example, QuantStudio™ 7 Real-Time PCR System (Thermo Fisher Scientific), or Lightcycler (Roche Molecular Biochemicals, Mannheim, Germany). In a preferred embodiment, the 5′ nuclease procedure is run on a real-time quantitative PCR device such as the QuantStudio™ 5 Real-Time PCR System. The system consists of a thermocycler, light-emitting diode (LED) and light wavelength filters, Complementary Metal-Oxide Semiconductor (CMOS) camera, and computer. The system amplifies samples in a 96-well format on a thermocycler. The RT-PCR may be performed in triplicate wells with an equivalent of 2 ng RNA input per 10 μl-reaction volume. During amplification, LED-induced fluorescent signal is collected in real-time, e.g. through fibre optics cables for all wells, and detected at the CMOS. The system includes software for running the instrument and for analysing the data.

In a particular embodiment, the RT-qPCR is carried out using primers capable of selectively hybridising to the target gene transcripts in the panel of genes.

In a particular embodiment, the level of RNA transcript for each gene has been normalised, such as by reference to the transcript level of at least one reference gene.

In a particular embodiment, the RT-qPCR is carried out on total RNA extracted from one or more slices or sections of the cancer sample.

In a particular embodiment the RNA transcript expression levels are measured using RT-PCR and the cycle threshold (Ct) level determined. The Ct level refers to the number of rounds or cycles of PCR that have been carried out before a certain threshold of amplification product has been produced. In a real-time PCR assay a positive reaction is detected by accumulation of a signal, typically a fluorescent signal. The Ct is defined as the number of cycles required for the signal to cross the threshold which is typically the background level. Sometimes Ct is referred to as Cq (quantification cycle). Ct levels are inversely proportional to the amount of target nucleic acid in the sample.

Current PCR instruments will collect the fluorescent data during each cycle and will be able to calculate the Ct value.

The measured Ct levels can then be normalised using the Ct of the reference gene(s) to get delta Ct (dCt). The delta Ct is the normalised Ct level which corresponds to the difference between Ct of the test gene and the Ct of the reference gene (or if more than one reference gene is used in the experiment the average of the reference genes). Suitably, gene expression measurements can be normalized relative to the mean of one or more (e.g., 2, 3, 4, 5, or more) reference genes.

Levels of proteins in a tumour sample can be used as a surrogate measure of RNA transcript expression levels. Levels of proteins in a tumour sample can be determined by any known techniques in the art, e.g., HPLC, mass spectrometry, or using antibodies specific to selected proteins (e.g., IHC, ELISA, etc.). Normalisation against reference proteins can then be carried out in a similar fashion as for RNA detection or as a ratio to references or by other standard approaches.

Real time PCR is compatible both with quantitative competitive PCR, where internal competitor for each target sequence is used for normalisation, and with quantitative comparative PCR using a normalisation gene contained within the sample, or a housekeeping gene for RT-PCR. For further details see, e.g. Held et al., Genome Research 6:986-994 (1996).

Other suitable techniques that can be employed include digital PCR or Serial analysis of gene expression (SAGE). For more details see, e.g. Velculescu et al., Science 270:484-487 (1995); and Velculescu et al., Cell 88:243-51 (1997).

### Gene Expression Analysis by Nucleic Acid Sequencing

Nucleic acid sequencing technologies can also be used to detect gene expression levels. The premise is that the number of times a cDNA sequence is detected in a sample is directly related to the relative expression of the mRNA corresponding to that sequence. These methods are sometimes referred to by the term Digital Gene Expression (DGE) to reflect the discrete numeric property of the resulting data. Early methods applying this principle were Serial Analysis of Gene Expression (SAGE) and Massively Parallel Signature Sequencing (MPSS). See, e.g., S. Brenner, et al., Nature Biotechnology 18(6):630-634 (2000). More recently, the advent of “next-generation” sequencing technologies has made DGE simpler, higher throughput, and more affordable. One such example is RNA sequencing (“RNA-seq”), in which cDNA molecules are synthesised from RNA, sequenced at a high throughput, and aligned to a reference standard. RNA expression can thus be determined by the number of aligned reads in a highly sensitive manner. Moreover, individual transcript splice variants can be identified and quantified, and genetic mutations or variants, such as single nucleotide polymorphisms (SNPs) determined.

### Data Analysis

In certain embodiments the expression level of the biomarker(s) can be compared to that detected in control cell(s), which may be obtained from non-cancerous tissue from the same or a different individual. Suitable controls include non-cancer cells from the same tissue or lineage. Comparison can be performed on test and reference samples measured concurrently or at temporally distinct times. An example of the latter is the use of compiled expression information, e.g., a sequence database, which assembles information about expression levels of the biomarker(s).

In some embodiments, the expression of one or more reference (sometimes called “housekeeping”) genes or proteins is also obtained for use in normalising the expression of the test genes/proteins. As used herein, “reference genes or proteins” refers to the genes or proteins whose expression is used to calibrate or normalise the measured expression of the test protein/gene of interest. The normalisation ensures accurate comparison of expression of a test biomarker between different samples. For this purpose, reference genes/proteins known in the art can be used. Examples of reference genes/proteins against which the biomarker expression levels can be normalised include but are not limited to: GAPDH and/or actin.

Increases or decreases in expression of the biomarkers disclosed herein can be determined based upon percent or fold changes over expression in normal cells, reference cells or normalised against one or more reference or housekeeping biomarkers. Increases may be of 10, 20, 30, 40, 50, 60, 70, 80, 90, 100, 120, 140, 160, 180, or 200% relative to expression levels in normal cells. Alternatively, fold increases may be of 1, 1.5, 2, 2.5, 3, 3.5, 4, 4.5, 5, 5.5, 6, 6.5, 7, 7.5, 8, 8.5, 9, 9.5, or 10-fold over expression levels in normal cells. Decreases may be of 10, 20, 30, 40, 50, 55, 60, 65, 70, 75, 80, 82, 84, 86, 88, 90, 92, 94, 96, 98, 99 or 100% relative to expression levels in normal cells. For example, a 2-fold increase or decrease is a useful measure for determining whether or not the expression level is low or high. Suitably, the threshold level of expression for making a forecast or call (e.g. whether a patient will likely respond to treatment with a PRMT5 inhibitor) based on the methods of the invention can be determined empirically using clinical samples.

### Medical Uses and Methods of Treatment

According to a fourth aspect of the invention there is provided a PRMT5 inhibitor or a pharmaceutical composition comprising a PRMT5 inhibitor for use in treating a patient with cancer whose cancer cells express greater than normal levels of E2F1 biomarker and/or reduced levels of pRb biomarker compared to normal.

In a variation of the fourth aspect of the invention there is provided a method of treating a patient with cancer whose cancer cells express greater than normal levels of E2F1 biomarker and/or reduced levels of pRb biomarker compared to normal, comprising administering to said patient an effective amount of a PRMT5 inhibitor or a pharmaceutical composition comprising a PRMT5 inhibitor.

In a variation of the fourth aspect of the invention there is provided a method of treating a patient with cancer comprising:
(i) determining the expression level of total E2F1 biomarker and/or pRb biomarker in a cancer cell sample of the patient,
(ii) comparing the levels determined in step (i) with the expression levels in normal cells; and
(iii) if the patient's cancer cells express greater than normal levels of E2F1 biomarker and/or reduced levels of pRb biomarker compared to normal the patient is administered an effective amount of a PRMT5 inhibitor or a pharmaceutical composition comprising a PRMT5 inhibitor.

In a variation of the fourth aspect of the invention there is provided the use of a PRMT5 inhibitor in the manufacture of a medicament for use in treating a patient with cancer whose cancer cells express greater than normal levels of E2F1 biomarker and/or reduced levels of pRb biomarker compared to normal.

Suitably, the patient whose cancer cells express greater than normal levels of E2F1 biomarker and/or reduced levels of pRb biomarker compared to normal has been identified according to the first or second aspect of the invention.

In a particular embodiment, the expression level of E2F1 biomarker is total E2F1 protein level.

In a particular embodiment, the expression level of E2F1 biomarker is E2F1 transcript level.

In a particular embodiment, the expression level of pRb biomarker is pRb protein level.

In a particular embodiment, the expression level of pRb biomarker is RB1 transcript level.

According to a fifth aspect of the invention there is provided a PRMT5 inhibitor for use in treating a pRb defective cancer or for use in treating a cancer whose cells express reduced levels of pRb biomarker compared to normal.

In a variation of the fifth aspect of the invention there is provided a method of treating a patient with a pRb defective cancer or whose cancer cells reduced levels of pRb biomarker compared to normal, comprising administering to said patient an effective amount of a PRMT5 inhibitor or a pharmaceutical composition comprising a PRMT5 inhibitor.

In a variation of the fifth aspect of the invention there is provided the use of a PRMT5 inhibitor in the manufacture of a medicament for use in treating a patient with a pRb defective cancer or whose cancer cells reduced levels of pRb biomarker compared to normal.

Suitably, the patient whose cancer cells express reduced levels of pRb biomarker compared to normal has been identified according to the first or second aspect of the invention.

In a particular embodiment, the expression level of pRb biomarker is pRb protein level.

In a particular embodiment, the expression level of pRb biomarker is RB1 protein level.

In a particular embodiment, patient whose cancer cells express reduced levels of pRb biomarker compared to normal has been identified by virtue of the cancer cells comprising one or more mutations in RB1 gene that result in reduced (including null) expression of pRb.

In particular embodiments, the one or more mutations in RB1 gene that result in reduced (including null) expression of pRb are selected from: E137X, R251X, R255X, R320X, R358X, R445X, R455X, R467X, R552X, R556X, R579X, R787X, R661W, C712R.

In a variation of the fourth aspect of the invention there is provided a method of treating a patient with cancer comprising:
(i) determining whether the RB1 gene in the cancer cells in a cancer cell sample of the patient comprise one or more mutations that result in reduced expression of pRb;
(ii) wherein if the RB1 gene in the cancer cells comprise one or more mutations that result in reduced expression of pRb the patient is administered an effective amount of a PRMT5 inhibitor or a pharmaceutical composition comprising a PRMT5 inhibitor.

In particular embodiments of the third or fourth aspects of the invention, the PRMT5 inhibitor is for use in a method of treating a cancer whose cells express greater than normal levels of E2F1 biomarker and reduced levels of pRb biomarker compared to normal. As explained above, a reduced levels of pRb biomarker compared to normal can be determined by detecting for the presence of one or more mutations in RB1 gene that result in reduced expression of pRb.

Any therapeutic agent capable of inhibiting PRMT5 can be employed in the fourth or fifth aspects of the invention. In particular embodiments, the PRMT5 inhibitor can be selected from the group consisting of: an antibody, an RNA interference molecule (such as microRNNmiRNA, small interfering RNA/siRNA or short-hairpin RNA/shRNA), an antisense oligonucleotide (ASO) or a small molecule compound.

Suitably, the PRMT5 inhibitor for use in the third or fourth aspect of the invention is in the context of a pharmaceutical composition comprising the PRMT5 inhibitor and at least one pharmaceutically acceptable component.

### PRMT5 Inhibitors

Any therapeutic agent capable of inhibiting PRMT5 can be employed in the fourth or fifth aspects of the invention. In particular embodiments, the PRMT5 inhibitor can be selected from the group consisting of: an antibody, an RNA interference molecule (such as microRNA/miRNA, small interfering RNA/siRNA or short-hairpin RNA/shRNA), an antisense oligonucleotide (ASO) or a small molecule compound.

In particular embodiments of any of the aspects disclosed herein, the PRMT5 inhibitor is a small molecule compound or a large molecule biologic.

Suitably, the PRMT5 inhibitor is selected from the group consisting of: an antibody, a peptide, a nucleic acid, a small molecule compound, an RNA inhibitory molecule (RNAi) and an antisense oligonucleotide (ASO).

In a particular embodiment, the PRMT5 inhibitor or pharmaceutical composition thereof for use according to the fourth or fifth aspects of the invention causes a reduction in functional activity of PRMT5 or expression levels of PRMT5.

### Nucleic Acid Inhibitors

In particular embodiments, the PRMT5 inhibitor for use in the invention is a nucleic-acid based therapeutic that comprise nucleic acid or nucleotides. By way of example, said nucleic acid therapeutic could be or comprises a dsRNA molecule, a RNAi molecule, a miRNA molecule, a ribozyme, a shRNA molecule, an antisense oligonucleotide (ASO), a guide RNA (gRNA) or a siRNA molecule.

RNAi and ASO molecules are particularly suitable for inhibiting the expression of PRMT5. The use of these approaches to down-regulate gene expression is now well-established in the art.

In a particular embodiment, the PRMT5 inhibitor is an RNAi.

In a particular embodiment, the PRMT5 inhibitor is an ASO.

The PRMT5 inhibitor for use in the invention could also be a nucleic acid-based molecule capable of inhibiting mRNA of PRMT5. There are many different types of nucleic acid-based molecules that can inhibit translation of an mRNA and/or decrease the stability of the RNA. Such an RNA inhibitor is preferably an RNAi molecule specific for PRMT5 mRNA; shRNA molecule specific for PRMT5 mRNA; or an antisense oligonucleotide (AON) specific for PRMT5 mRNA.

Thus, according to another embodiment, the PRMT5 inhibitor is or comprises a nucleic acid molecule capable of inhibiting mRNA of PRMT5.

### Antibody

The PRMT5 inhibitor could also be a large molecule biologic, such as an antibody or an antibody fragment. In a particular embodiment, the PRMT5 inhibitor is a monoclonal antibody. In a particular embodiment, the PRMT5 inhibitor is a monoclonal antibody fragment. In a particular embodiment, the PRMT5 inhibitor is a polyclonal antibody. In a particular embodiment, the PRMT5 inhibitor is an intrabody.

An antibody is an immunoglobulin molecule capable of specific binding to a target, such as a carbohydrate, polynucleotide, lipid, polypeptide, etc., through at least one antigen recognition site, located in the variable domain of the immunoglobulin molecule.

An “intact antibody” typically refers to a Y-shaped tetrameric protein comprising two heavy (H) and two light (L) polypeptide chains held together by covalent disulfide bonds and non-covalent interactions. Each light chain is composed of one variable domain (VL) and one constant domain (CL). Each heavy chain comprises one variable domain (VH) and a constant region, which in the case of IgG, IgA, and IgD antibodies, comprises three domains termed CH1, CH2, and CH3 (IgM and IgE have a fourth domain, CH4). In IgG, IgA, and IgD classes the CH1 and CH2 domains are separated by a flexible hinge region, which is a proline and cysteine rich segment of variable length (from about 10 to about 60 amino acids in various IgG subclasses).

The variable domains in both the light and heavy chains are joined to the constant domains by a “J” region of about 12 or more amino acids and the heavy chain also has a “D” region of about 10 additional amino acids. Each class of antibody further comprises inter-chain and intra-chain disulfide bonds formed by paired cysteine residues. The heavy chain variable region (YH) and light chain variable region (YL) can each be further subdivided into regions of hypervariability,

termed complementarity determining regions (CDRs), interspersed with regions that are more conserved, termed framework regions (FR). Each YH and YL, comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

As used herein, the term “antibody” includes, by way of example, both naturally occurring and non-naturally occurring intact antibodies, such as polyclonal, multiclonal or monoclonal antibodies, as well as chimeric antibodies, humanized and primatized antibodies, CDR grafted antibodies, human antibodies, intrabodies, multi-specific antibodies, bispecific antibodies, monovalent antibodies, multivalent antibodies, anti-idiotypic antibodies and synthetic antibodies, but also, unless otherwise specified, any antigen-binding portion thereof that competes with the intact antibody for specific binding, fusion proteins comprising an antigen-binding portion, and any other modified configuration of the immunoglobulin molecule that comprises an antigen recognition site.

It has been shown that the antigen-binding function of an antibody can be performed by portions of a full-length antibody. Antigen-binding portions of an antibody (also called an “antigen-binding fragment”) refers to one or more fragments of an antibody that retain the ability to specifically bind to an antigen (e.g., PRMT5) bound by the whole antibody.

Antigen-binding portions include, for example, Fab, Fab′, F(ab′)2, F(ab′) fragments, Fd, Fv, domain antibodies (dAbs, e.g., shark and camelid antibodies), portions including complementarity determining regions (CDRs), single chain variable fragment antibodies (e.g. scFv, scFvFc and bis-scFv), minibodies, maxibodies, intrabodies, diabodies, triabodies, tetrabodies, v-NAR and polypeptides that contain at least a portion of an immunoglobulin that is sufficient to confer specific antigen binding to the polypeptide. Depending on the antibody amino acid sequence of the constant region of its heavy chains, immunoglobulins can be assigned to different classes. There are five major classes (i.e., isotypes) of immunoglobulins: IgA, IgD, IgE, IgG, and IgM, and several of these may be further divided into subclasses (subtypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known. Unless dictated otherwise by contextual constraints the term further comprises all classes and subclasses of antibodies. Heavy-chain constant domains that correspond to the different classes of antibodies are typically denoted by the corresponding lower-case Greek letter α, δ, ε, γ, and μ, respectively. Light chains of the antibodies from any vertebrate species can be assigned to one of two clearly distinct types, called kappa (κ) and lambda (λ), based on the amino acid sequences of their constant domains.

Although the two domains of the Fv portion, VL and VH, are coded for by separate genes, they can be joined, using recombinant methods, by a synthetic linker that enables them to be made as a single protein chain in which the VL and VH regions pair to form monovalent molecules (known as single chain Fv (scFv)); see e.g., Bird et al. Science 242:423-426 (1988) and Huston et al. Proc. Natl. Acad. Sci. USA 85:5879-5883 (1988)). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. Proc. Natl. Acad. Sci. USA 90:6444-6448, 1993; Poljak et al., Structure. 2:1121-1123, 1994).

The antibodies may be murine, rat, human, or any other origin (including chimeric or humanized antibodies). In some embodiments, the antibody is a monoclonal antibody. In some embodiments, the antibody is a human or humanized antibody. A non-human antibody may be humanized by recombinant methods to reduce its immunogenicity in man.

The term “monoclonal antibody” (“mAb”) refers to a non-naturally occurring preparation of antibody molecules of single molecular composition, i.e., antibody molecules whose primary sequences are essentially identical. A Mab is highly specific, being directed against a single antigenic site/epitope. A mAb is an example of an isolated antibody. MAbs may be produced by hybridoma, recombinant, transgenic or other techniques known to those skilled in the art. The modifier “monoclonal” indicates the character of the antibody as being obtained from a substantially homogeneous population of antibodies and is not to be construed as requiring production of the antibody by any particular method. For example, the monoclonal antibodies to be used in accordance with the present disclosure may be made by the hybridoma method first described by Kohler and Milstein (Nature 256:495,1975) or may be made by recombinant DNA methods such as described in U.S. Pat. No. 4,816,567. The monoclonal antibodies may also be isolated from phage libraries generated using the techniques described in McCafferty et al., (Nature 348:552-554, 1990), for example.

A “human” antibody (HuMAb) refers to an antibody having variable regions in which both the framework and CDR regions are derived from human germline immunoglobulin sequences. Furthermore, if the antibody contains a constant region, the constant region is also derived from human germline immunoglobulin sequences.

The human antibodies may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human” antibodies and “fully human” antibodies are used synonymously. This definition of a human antibody specifically excludes a humanized antibody comprising non-human antigen binding residues.

As used herein, a “humanized antibody” refers to an antibody in which some, most or all of the amino acids outside the CDR domains of a non-human antibody are replaced with corresponding amino acids derived from human immunoglobulins. In some embodiments, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. The humanized antibody may comprise residues that are found neither in the recipient antibody nor in the imported CDR or framework sequences but are included to further refine and optimize antibody performance. In one embodiment of a humanized form of an Ab, some, most or all the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible provided they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody or vice versa. The term also encompasses an antibody comprising a V region from one individual from one species (e.g., a first mouse) and a constant region from another individual from the same species (e.g., a second mouse).

An “intrabody” refers to an antibody that has been designed to be expressed intracellularly and can be directed to a specific target antigen present in various subcellular locations including the cytoplasm, nucleus and endoplasmic reticulum through in frame fusion with intracellular localization peptide sequences. It has been identified as a new class of therapeutic molecule (Chen et al., Human Gene Therapy. 5 (5): 595-601, 1994). Although intrabodies can be expressed in different forms, the most commonly used format is a scFv due to their mall size. Antibody fragments, typically in scFv format, are cloned into a specific targeting vector allowing expression of the intrabody in the nucleus, cytoplasm or ER. The intrabody gene is expressed inside the target cell after transfection with an expression plasmid or viral transduction with a recombinant virus. It has been found that the usual vector-, promoter- and transfection systems for heterologous expression can be employed to express the intrabody in the cell of interest.

Generally, the term “epitope” refers to the area or region of an antigen to which an antibody specifically binds, i.e., an area or region in physical contact with the antibody. Thus, the term “epitope” refers to that portion of a molecule capable of being recognized by and bound by an antibody at one or more of the antibody's antigen-binding regions.

An antibody that “specifically binds” to an epitope is a term well understood in the art, and methods to determine such specific binding are also well known in the art. A molecule is said to exhibit “specific binding” if it reacts or associates more frequently, more rapidly, with greater duration and/or with greater affinity with a particular cell or substance than it does with alternative cells or substances. For example, an antibody that specifically binds to an PRMT5 epitope is an antibody that binds this epitope with greater affinity, avidity, more readily, and/or with greater duration than it binds to other PRMT5 epitopes or non-PRMT5 epitopes. It is also understood by reading this definition, for example, that an antibody which specifically binds to a first target may or may not specifically or preferentially bind to a second target. As such, “specific binding” does not necessarily require (although it can include) exclusive binding.

An antibody to PRMT5, for example, may be made by any method known in the art. General techniques for production of human and mouse antibodies are known in the art and/or are described herein. For example, see Harlow and Lane (1988) “Antibodies: A Laboratory Manual”, Cold Spring Harbor Laboratory Press, Cold Spring Harbor, NJ.

In some embodiments, antibodies may be made recombinantly and expressed using any method known in the art. In some embodiments, antibodies may be prepared and selected by phage display technology. See, for example, U.S. Pat. Nos. 5,565,332; 5,580,717; 5,733,743; and 6,265, 50; and Winter et al., (Annu. Rev. Immunol. 12:433-455, 1994). Alternatively, the phage display technology (McCafferty et al., Nature 348:552-553, 1990) can be used to produce human antibodies and antibody portions in vitro, from immunoglobulin variable (V) domain gene repertoires from unimmunized donors.

In particular embodiments, the antibody for use in the invention is selected from: a monoclonal, human, humanised, Fab, Fab′, F(ab′)2, F(ab′), Fd, Fv, dAb, intrabody, scFV and VHH antibody.

Antibody and nucleic-acid technology molecules (such as RNAi and ASO) technologies are suitably advanced that the person skilled in the art would be able to make an antibody or antibody-derived molecule or a nucleic-acid technology molecule that could inhibit PRMT5.

### Small Molecule PRMT5 Inhibitor Compounds

In a particular embodiment, the PRMT5 inhibitor is a small molecule compound.

A “small molecule” as used herein, is an organic molecule that is less than about 5 kilodaltons (KDa) in mass. In some embodiments, the small molecule is less than about 3 KDa, or less than about 2 KDa, or less than about 1.5 KDa, or less than about 1 KDa. Most small molecule compounds are less than around 800 daltons (Da). In some embodiments, the small molecule is less than about 800 Da, less than about 600 Da, less than about 500 Da, less than about 400 Da, less than about 300 Da, less than about 200 Da, or less than about 100 Da. Often, a small molecule has a mass of at least 50 Da. In some embodiments, a small molecule is non-polymeric. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups.

PRMT5 inhibitor compounds with distinct chemophores are known. Fereira de Freitas et al., (Molecules. 244492, 2019) reviews some of the PRMT5 inhibitors, recites their structures and outlines their mechanism of action.

The following table lists some of the patent publication filed by various pharmaceutical companies, and others, directed to PRMT5 inhibitors which could be used in the invention.

| | |
|---|---|
| Epizyme Inc | WO 2016/022605 |
| & GSK | |
| Epizyme Inc | WO 2015/200680, WO 2015/200677, WO 2014/100730, |
| | WO 2014/100734, WO 2014/100719, WO 2014/100764, |
| | WO 2014/100695, WO 2014/100716, WO 2014/062720 |
| Eli Lilly | WO 2016/178870 |
| Merck Sharp & | WO 2021/126728, WO 2020/033285, WO 2020/033288, |
| Dohme | WO 2019/094311, WO 2021/126731, WO 2020/033282, |
| | WO 2021/126732, WO 2019/094312, WO 2021/126729, |
| | WO 2020/033284 |
| Prelude | WO 2018/160855, WO 2018/075601, WO 2019/178368, |
| Therapeutics | WO 2018/152548, WO 2019/084470, WO 2018/160824, |
| | WO 2019/032859, WO 2019/085818, WO 2018/085833, |
| | WO 2018/152501, WO 2021/055797, WO 2020168125 |
| Janssen | WO 2017/153186, WO 2018/065365, WO 2017/032840, |
| Pharmaceutica | WO 2019/110734 |
| Lupin Limited | WO 2021/111322, WO 2019/116302, WO 2020/250123 |
| Bayer Akt | WO 2019/002074 |
| Angex Pharma | WO 2019/112719, WO 2020/243178 |
| SK Biopharma | WO 2021/080359, WO 2021/066578 |
| Amgen | WO 2021/163344 |
| Pfizer | WO 2021/140427 |
| Others | WO 2021/207052, WO 2021/088992, WO 2020/198323, |
| | WO 2020/259478, WO 2020/205660, WO 2018/161922, |
| | WO 2019/165189, WO 2018/081451, WO 2019/173804, |
| | WO 2017/153513, WO 2016/145150, WO 2019/180628, |
| | WO 2021/050915, WO 2014/145214, WO 2017/153515, |
| | WO 2021/202480, WO 2019/180631, WO 2016/034675, |
| | WO 2017/153518, WO 2019/102494, WO 2016/034673, |
| | WO 2021/068953, WO 2017/218802, WO 2020/205867, |
| | WO 2016/034671, WO 2011/079236 |

Compound 208 in WO 2014/100719 (Epizyme) is GSK3326595 (pemrametostat).

The compound of Example 2 in WO 2016/178870 (Eli Lilly) is LLY-283

Compound 80 in WO 2017/032840 (Janssen Pharmaceuticals) is JNJ-64619178.

See also Fereira de Freitas et al., (Molecules. 24:4492, 2019).

Other suitable PRMT5 inhibitors include:
(1) WO 2018/167269 (Argonaut Therapeutics Limited) which disclose compounds of formula I, or a salt, solvate or hydrate thereof,

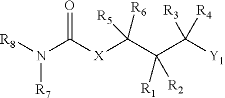

wherein,
R₁, R₃, R₄, R₅ and R₆ are each independently selected from hydrogen and C₁₋₃ alkyl;
R₂ is selected from hydrogen and R₁₄;
X is O or NR₉, where Re is hydrogen or a C₁₋₃ alkyl;
Y₁ is a group selected from one of formula A and B,

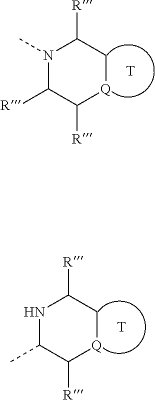

where each R′″ is independently selected from H and C₁₋₃ alkyl;
Q is C or N;
T is selected from a fused phenyl group and a fused 5- or 6-membered heteroaryl group, wherein each group is optionally substituted with one or more substituents selected from halo and C₁₋₃ alkyl; and
R₇ and R₈ are taken together with the intervening nitrogen atom to form a 3-12 membered heterocycloalkyl ring, wherein the 3-12 membered heterocycloalkyl ring is optionally substituted with one or more R₁₀; and/or optionally fused to one or more C₆₋₁₂ aryl, C₅₋₁₂ heteroaryl, C₃₋₈ cycloalkyl and 3-12 membered heterocycloalkyl rings, wherein each fused C₆₋₁₂ aryl, C₅₋₁₂ heteroaryl, C-6 cycloalkyl and 3-12 membered heterocycloalkyl ring is optionally substituted with one or more R₁₄;
R₁₀ is selected from a group of the formula L₁-L₂-R₁₁ or L₂-L₁-R₁₁, where L₁ is a linker of the formula —[CR₁₂R₁₃]ₙ—, where n is an integer of from 0 to 3 and R₁₂ and R₁₃ are in each instance each independently selected from H and C₁ to C₂ alkyl,
where L₂ is absent or a linker that is selected from O, S, SO, SO₂, N(R′), C(O), C(O)O, [O(CH₂)ᵣ]ₛ, [(CH₂)ᵣO]ₛ, OC(O), CH(OR′), C(O)N(R′), N(R′)C(O), N(R′)C(O)N(R′), SO₂N(R′) or N(R′)SO₂, where R′ and R″ are each independently selected from hydrogen and a C₁ to C₂ alkyl, and where r is 1 or 2 and s is 1 to 4,
R₁₁ is independently selected from hydrogen, CN, NO₂, hydroxyl, ═O, halogen, C₁₋₆ haloalkyl, C₁₋₆ haloalkoxy, C₁₋₆ alkyl, O—C₁₋₆ alkyl, C₃₋₆ cycloalkyl, C₆₋₁₂ aryl, C₅₋₁₂ heteroaryl, 3-10 membered heterocycloalkyl, —C(═O)R^{d}, —C(═O)OR^{d}, —C(═O)NR^{e}R^{d}, —C(O)C(═O)R^{d}, —NR^{e}R^{d}, —NR^{e}C(═O)R^{d}, —NR^{e}C(═O)OR^{d}, —NR^{e}C(═O)NR^{e}R^{d}, —NR^{e}S(═O)₂R^{d}, —NR^{e}S(═O)₂NR^{e}R^{d}, —OR^{d}, —SR^{d}, —OC(═O)R^{d}, —OC(═O)NR^{e}R^{d}, —OC(═O)OR^{d}, —S(═O)₂R^{d}, —S(═O)R^{d}, —OS(═O)R^{d}, —OS(═O)₂R^{d}, —OS(═O)₂OR^{d}, —S(═O)NR^{e}R^{d}, —OS(═O)₂NR^{e}R^{d}, and —S(═O)₂NR^{e}R^{d}, wherein, where R₁₁ is independently selected from C₃₋₆ cycloalkyl, C₆₋₁₂ aryl, C₅₋₁₂ heteroaryl and 3-10 membered heterocycloalkyl, each C₃₋₆ cycloalkyl, C₆₋₁₂ aryl, C₅₋₁₂ heteroaryl and 3-10 membered heterocycloalkyl is optionally substituted with one or more R₁₄;
each R^{a} and R^{b} is independently selected from hydrogen and C₁₋₆ alkyl;
each R^{d} is independently selected from hydrogen, hydroxyl, halogen, CN, C₁₋₆ haloalkyl, 3-7 membered heterocycloalkyl, C₃₋₆ cycloalkyl, C₁₋₆ alkyl, O—C₁₋₆ alkyl and C₆₋₁₁ aryl, wherein said C₁₋₆ alkyl, C₆₋₁₁ aryl, 3-7 membered heterocycloalkyl and C₃₋₆ cycloalkyl are optionally substituted with one or more groups selected from hydroxyl, ═O, halogen, CN, COR^{a}, NR^{a}R^{b}, C₁₋₆ haloalkyl, C cycloalkyl, C₆₋₁₁ aryl, 3-7 membered heterocycloalkyl, C₁₋₆ alkyl and O—C₁₋₆ alkyl;
each R^{e} is independently selected from hydrogen, hydroxyl, halogen, CN, C₁₋₆ haloalkyl, C₃₋₆ cycloalkyl, C₁₋₆ alkyl and O—C₁₋₆ alkyl; or
R^{e} and R^{d}, when attached to the same atom, together with the atom to which they are attached form a 3-7 membered heterocycloalkyl ring, optionally substituted with one or more substituent selected from hydroxyl, ═O, halogen, CN, COR^{a}, NR^{a}R^{b}, C₁₋₆ haloalkyl, C₃₋₆ cycloalkyl, C₆₋₁₁ aryl, 3-7 membered heterocycloalkyl, C₁₋₆ alkyl and O—C₁₋₆ alkyl; and
R¹⁴ is independently selected from halo, CN, NO₂, hydroxyl, ═O, halogen, C₁₋₆ haloalkyl, C₁₋₆ haloalkoxy, C₁₋₆ alkyl, O—C₁₋₆ alkyl, C₃₋₆ cycloalkyl, C₆₋₁₂ aryl, 5-6 membered heteroaryl, 3-7 membered heterocycloalkyl, C₁₋₆alkylC₆₋₁₂aryl, —C(═O)R^{d}, —C(═O)OR^{d}, —C(═O)NR^{e}R^{d}, —C(O)C(═O)R^{d}, —NR^{e}R^{d}, —NR^{e}C(═O)R^{d}, —NR^{e}C(═O)OR^{d}, —NR^{e}C(═O)NR^{e}R^{d}, —NR^{e}S(═O)₂R^{d}, —NR^{e}S(═O)₂NR^{e}R^{d}, —OR^{d}, —SR^{d}, —OC(═O)R^{d}, —OC(═O)NR^{e}R^{d}, —OC(═O)OR^{d}, —S(═O)₂R^{d}, —S(═O)R^{d}, —OS(═O)R^{d}, —OS(═O)₂R^{d}, —OS(═O)₂OR^{d}, —S(═O)NR^{e}R^{d}, —OS(═O)₂NR^{e}R^{d}, and —S(═O)₂NR^{e}R^{d}.
(2) WO 2018/167276 (Argonaut Therapeutics Limited) which disclose compounds of formula I, or a salt, solvate or hydrate thereof,

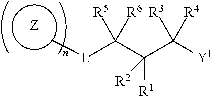

wherein,
Y¹ is a group selected from one of formula A and B,

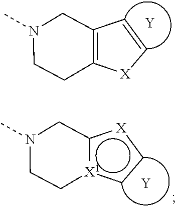

X is selected from O, S, CH and NR⁷;
X¹ is selected from C and N;
Y is selected from a fused aryl group and a fused heteroaryl group, where each group is optionally substituted with one or more R¹¹;
n is 1 and L is selected from —(CH₂)^{p}N(R^{a})C(O)—, —(CH₂)ₚC(O)N(R^{a})—, —(CH₂)ₚN(R^{a})S(O_{q})—, —(CH₂)ₚS(O_{q})N(R^{a})—, —(CH₂)ₚN(R^{b})C(O)N(R^{b})—, —(CH₂)ₚN(R^{c})C(O)O—, and —(CH₂)ₚOC(O)N(R^{c})—; or
n is 0 and L is selected from R^{d}(R^{e})NC(O)—, —R^{d}(R^{e})NC(O)N(R^{b})—, R^{d}(R^{e})NC(O)O—, R^{d}(R^{e})NS(O_{q}) and R^{d}(R^{e})N—;
p is a number selected from 0, 1, 2 and 3;
q is a number selected from 1 and 2;
Z is selected from C₆₋₁₁aryl optionally substituted by one or more R¹⁰, (C₇₋₁₆)alkylaryl optionally substituted by one or more R¹⁰, C₃₋₁₁ cycloalkyl optionally substituted by one or more R¹⁰, (C₄₋₁₇)cycloalkylalkyl optionally substituted by one or more R¹⁰, 3-15 membered heterocycloalkyl optionally substituted by one or more R¹⁰, 4-21 membered alkylheterocycloalkyl optionally substituted by one or more R¹⁰, 5-15 membered heteroaryl optionally substituted by one or more R¹⁰, and 6-21 membered alkylheteroaryl optionally substituted by one or more R¹⁰;
R¹ is selected from hydrogen, halogen, —NR^{e}R^{d}, OR^{f}, and C₁₋₆ alkyl optionally substituted with one or more R⁹;
R² is selected from hydrogen, halogen and C₁₋₆ alkyl optionally substituted with one or more R⁹;
R³, R⁴, R⁵ and R⁶ are independently selected from hydrogen, halogen and C₁₋₆ alkyl optionally substituted with one or more R⁹;
R⁷ is selected from hydrogen, hydroxyl, C₁₋₆ alkyl, C₁₋₆ haloalkyl, phenyl and C₃₋₆ cycloalkyl, wherein said C₁₋₆ alkyl, phenyl and C₃₋₆ cycloalkyl are optionally substituted by one or more substituents selected from hydroxyl, halogen, ═O, CN, COR^{a}, NR^{a}R^{b}, C₁₋₆ haloalkyl, C₃₋₆ cycloalkyl, C₆₋₁₁ aryl, 3-7 membered heterocycloalkyl, C₁₋₆ alkyl and O—C₁₋₆ alkyl;
each R⁹ is independently selected from hydrogen, hydroxyl, halogen, CN, C₁₋₆ haloalkyl, 3-7 membered heterocycloalkyl, C₃₋₆ cycloalkyl, C₁₋₆ alkyl, O—C₁₋₆ alkyl and phenyl, wherein said C₁₋₆ alkyl, phenyl, 3-7 membered heterocycloalkyl and C₃₋₆ cycloalkyl are optionally substituted with one or more groups selected from hydroxyl, ═O, halogen, CN, NR^{a}R^{b}, COR^{a}, C₁₋₆ haloalkyl, C₃₋₆ cycloalkyl, phenyl, 3-7 membered heterocycloalkyl, C₁₋₆ alkyl and O—C₁₋₆ alkyl;
each R¹⁰ is independently selected from hydrogen, hydroxyl, ═O, halogen, CN, C₁₋₆ haloalkyl, C₁₋₆ haloalkoxy, C₁₋₆ alkyl, O—C₁₋₆ alkyl, C₃₋₆ cycloalkyl, phenyl, 5-6 membered heteroaryl, 3-7 membered heterocycloalkyl, —C(═O)R^{d}, —C(═O)OR^{d}, —C(═O)NR^{e}R^{d}, —C(O)C(═O)R^{d}, —NR^{e}R^{d}, —NR^{e}C(═O)R^{d}, —NR^{e}C(═O)OR^{d}, —NR^{e}C(═O)NR^{e}R^{d}, —NR^{e}S(═O)₂R^{d}, —NR^{e}S(═O)₂NR^{e}R^{d}, —OR^{d}, —SR^{d}, —OC(═O)R^{d}, —OC(═O)NR^{e}R^{d}, —OC(═O)OR^{d}, —S(═O)₂R^{d}, —S(═O)R^{d}, —OS(═O)R^{d}, —OS(═O)₂R^{d}, —OS(═O)₂OR^{d}, —S(═O)NR^{e}R^{d}, —OS(═O)₂NR^{e}R^{d}, and —S(═O)₂NR^{e}R^{d}, where said C₃₋₆ cycloalkyl, C₁₋₆ alkyl, phenyl, 5-6 membered heteroaryl and 3-7 membered heterocycloalkyl are optionally substituted with one or more groups selected from hydroxyl, halogen, ═O, CN, C₁₋₆ haloalkyl, C₁₋₆ haloalkoxy, C₃₋₆ cycloalkyl, C₁₋₆ alkyl and O—C₁₋₆ alkyl;
R¹¹ is selected from hydrogen, hydroxyl, halogen, CN, NR^{a}R^{b}, C₁₋₆ haloalkyl, 3-7 membered heterocycloalkyl, C₃₋₆ cycloalkyl, C₁₋₆ alkyl, O—C₁₋₆ alkyl and phenyl, wherein said C₁₋₆ alkyl, phenyl, 3-7 membered heterocycloalkyl and C₃₋₆ cycloalkyl are optionally substituted with one or more groups selected from hydroxyl, ═O, halogen, CN, COR^{a}, NR^{a}R^{b}, C₁₋₆ haloalkyl, C₃₋₆ cycloalkyl, C₆₋₁₁ aryl, 3-7 membered heterocycloalkyl, C₁₋₆ alkyl and O—C₁₋₆ alkyl;
each R^{a}, R^{b} and R^{c} is independently selected from hydrogen and C₁₋₆ alkyl;
each R^{d} is independently selected from hydrogen, hydroxyl, halogen, CN, C₁₋₆ haloalkyl, 3-7 membered heterocycloalkyl, C₃₋₆ cycloalkyl, C₁₋₆ alkyl, O—C₁₋₆ alkyl and C₆₋₁₁ aryl, wherein said C₁₋₆ alkyl, C₆₋₁₁ aryl, 3-7 membered heterocycloalkyl and C₃₋₆ cycloalkyl are optionally substituted with one or more groups selected from hydroxyl, ═O, halogen, CN, COR^{a}, NR^{a}R^{b}, C₁₋₆ haloalkyl, C₃₋₆ cycloalkyl, C₆₋₁₁ aryl, 3-7 membered heterocycloalkyl, C₁₋₆ alkyl and O—C₁₋₆ alkyl;
each R^{e} is independently selected from hydrogen, hydroxyl, halogen, CN, C₁₋₆ haloalkyl, C₃₋₆ cycloalkyl, C₁₋₆ alkyl and O—C₁₋₆ alkyl; or
R^{e} and R^{d}, when attached to the same atom, together with the atom to which they are attached form a 3-7 membered heterocycloalkyl ring, optionally substituted with one or more substituent selected from hydroxyl, ═O, halogen, CN, COR^{a}, NR^{a}R^{b}, C₁₋₆ haloalkyl, C₃₋₆ cycloalkyl, C₆₋₁₁ aryl, 3-7 membered heterocycloalkyl, C₁₋₆ alkyl and O—C₁₋₆ alkyl; and
R^{f} is independently selected from hydrogen and C₁₋₆ alkyl optionally substituted with one or more substituents selected from hydroxyl, halogen, CN, COR^{a}, NR^{a}R^{b}, C₁₋₆ haloalkyl, C₃₋₆ cycloalkyl, phenyl, 3-7 membered heterocycloalkyl and O—C₁₋₆ alkyl.
(3) GB2108383.7 (Argonaut Therapeutics Limited) which discloses compounds of formula (1) or a deuterated form, salt, solvate, or hydrate thereof,

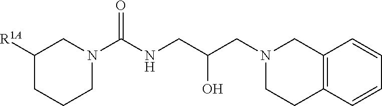

wherein:
R^{1A} is represented by formula (A1),

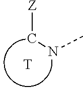

Z is ═O;
T taken together with the intervening carbon and nitrogen atoms (e.g. shown in formula (A1)) is selected from a monocyclic 5- to 7-membered heterocycloalkyl group, a fused bicyclic 6- to 10-membered heterocycloalkyl group and a bridged bicyclic 6- to 9-membered heterocycloalkyl group, wherein each of the monocyclic 5- to 7-membered heterocycloalkyl group, the fused bicyclic 6- to 10-membered heterocycloalkyl group and the bridged bicyclic 6- to 9-membered heterocycloalkyl group is optionally substituted with one or more R^{S1};
R^{S1} is selected from C₁₋₆ alkyl, C₂₋₆ alkenyl, C₂₋₆alkynyl, C₁₋₆alkoxy, C₃₋₁₂cycloalkyl, hydroxy, halo, CN and nitro, wherein the C₁₋₆alkyl, the C₂₋₆alkenyl, the C₂₋₆alkynyl and the C₃₋₁₂cycloalkyl is each optionally substituted with one or more R^{S2}; and
R^{S2} is selected from hydroxy, halo, CN and nitro.

Any of these PRMT5 compounds can be used in the present invention.

In particular embodiments, the PRMT5 inhibitor for use in the present invention is a small molecule compound selected from the group consisting of: GSK3326595 (pemrametostat), PF-6939999, JVNJ-64619178 (onametostat), LLY-283 and PRT543.

The PRMT5 inhibitor for use in the fourth or fifth aspects of the invention may be formulated as a pharmaceutical composition. The pharmaceutical composition may comprise at least one pharmaceutically-acceptable excipient. The dosage, route of administration and indeed treatment regime can be determined by the person of sill in the art.

The term “pharmaceutically-acceptable excipient” as used herein means one or more compatible solid or liquid fillers, diluents or encapsulating substances that are suitable for administration into a human. The term “excipient” denotes an organic or inorganic ingredient, natural or synthetic, with which the active ingredient is combined to facilitate the application. Types of suitable excipient are salts, buffering agents, wetting agents, emulsifiers, preservatives, compatible carriers, diluents, carriers, vehicles, supplementary immune potentiating agents such as adjuvants and cytokines that are well known in the art and are available from commercial sources for use in pharmaceutical preparations (see, e.g. Remington: The Science and Practice of Pharmacy with Facts and Comparisons: Drugfacts Plus, 20th Ed. Mack Publishing; Kibbe et al., (2000) Handbook of Pharmaceutical Excipients, 3rd Ed., Pharmaceutical Press; and Ansel et al., (2004) Pharmaceutical Dosage Forms and Drug Delivery Systems, 7th Ed., Lippencott Williams and Wilkins). Optionally, the pharmaceutical compositions contain one or more other therapeutic agents or compounds. Suitable pharmaceutically acceptable excipients are relatively inert and can facilitate, for example, stabilisation, administration, processing or delivery of the active compound/agent into preparations that are optimised for delivery to the body, and preferably directly to the site of action.

The pharmaceutical compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use.

When administered, the PRMT5 inhibitor is administered in pharmaceutically acceptable preparations/compositions.

Administration may be enteral (e.g. oral), i.e., substance is given via the gastrointestinal tract, or parenteral, i.e., substance is given by other routes than the digestive tract such as by injection. Large biologic molecules or nucleic acid molecules (such as certain vaccines) are typically administered parenterally by injection.

Pharmaceutical compositions for parenteral administration (e.g. by injection), include aqueous or non-aqueous, isotonic, pyrogen-free, sterile liquids (e.g. solutions, suspensions), in which the active ingredient/agent is dissolved, suspended, or otherwise provided (e.g. in a liposome or other microparticulate). Such liquids may additionally contain one or more pharmaceutically acceptable carriers, such as anti-oxidants, buffers, stabilisers, preservatives, suspending agents, and solutes that render the formulation isotonic with the blood (or other relevant bodily fluid) of the intended patient. In particular embodiments, the composition may be lyophilised to provide a powdered form that is ready for reconstitution as and when needed. When reconstituted from lyophilised powder the aqueous liquid may be further diluted prior to administration. For example, diluted into an infusion bag containing 0.9% sodium chloride injection, USP, or equivalent, to achieve the desired dose for administration. In particular embodiments, such administration can be via intravenous infusion using an intravenous (IV) apparatus.

Suitably, the PRMT5 inhibitor agent is formulated in accordance with routine procedures as pharmaceutical compositions adapted for intravenous administration to human beings. Typically, the active agent for IV administration is in solution, e.g. in sterile isotonic aqueous buffer. Where necessary, the compositions can also include a solubilizing agent. Compositions for IV administration can optionally include a local anaesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule. Where the PRMT5 inhibitor agent is to be administered by infusion, it can be dispensed, for example, with an infusion bottle containing sterile pharmaceutical grade water or saline. Where the PRMT5 inhibitor agent is administered by injection, an ampoule of sterile water for injection or saline can be provided so that the ingredients can be mixed prior to administration.

Compositions for oral delivery can be in the form of tablets, lozenges, aqueous or oily suspensions, granules, powders, emulsions, capsules, syrups, or elixirs, for example, prepared by conventional means with pharmaceutically acceptable excipients such as binding agents (e.g., pre-gelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavouring, colouring and sweetening agents as appropriate. Oral compositions can include standard vehicles such as mannitol, lactose, starch, magnesium stearate, sodium saccharine, cellulose, magnesium carbonate, and the like.

Compositions for use in accordance with the present invention can be formulated in conventional manner using one or more physiologically acceptable excipients. Thus, the PRMT5 inhibitor agent and optionally another therapeutic or prophylactic agent and their physiologically acceptable salts and solvates can be formulated into pharmaceutical compositions for administration by inhalation or insufflation (either through the mouth or the nose) or oral, parenteral or mucosal (such as buccal, vaginal, rectal, sublingual) administration. In a particular embodiment, local or systemic parenteral administration is used.

The pharmaceutical compositions for use the treatment methods of the invention are for administration in an effective amount. An “effective amount” is the amount of a composition that alone, or together with further doses, produces the desired response.

Suitably, the PRMT5 inhibitor agent can be administered as a pharmaceutical composition in which the pharmaceutical composition comprises between 0.1-1 mg, 1-10 mg, 10-50 mg, 50-100 mg, 100-500 mg, or 500 mg to 5 g of the PRMT5 inhibitor agent.

The preparation of a suitable pharmaceutical composition of the drug and the dosage to administer to a subject is within the capabilities of a person of skill in the art.

### Cancers

The various aspects of the invention that use cancer cells, or are directed to methods or uses for treating cancer, apply to any cancer. Suitably, the cancer is selected from the group consisting of: leukaemia, lymphoma, multiple myeloma, lung cancer, liver cancer, breast cancer, head and neck cancer, neuroblastoma, thyroid carcinoma, skin cancer (including melanoma), oral squamous cell carcinoma, urinary bladder cancer, Leydig cell tumour, biliary cancer, such as cholangiocarcinoma or bile duct cancer, brain cancer, pancreatic cancer, colon cancer, colorectal cancer and gynaecological cancers, including ovarian cancer, endometrial cancer, fallopian tube cancer, uterine cancer and cervical cancer, including epithelia cervix carcinoma. In suitable embodiments, the cancer is leukaemia and can be selected from the group consisting of acute lymphoblastic leukaemia, acute myelogenous leukaemia (also known as acute myeloid leukaemia or acute non-lymphocytic leukaemia), acute promyelocytic leukaemia, acute lymphocytic leukaemia, chronic myelogenous leukaemia (also known as chronic myeloid leukaemia, chronic myelocytic leukaemia or chronic granulocytic leukaemia), chronic lymphocytic leukaemia, monoblastic leukaemia and hairy cell leukaemia. In further preferred embodiments, the cancer is acute lymphoblastic leukaemia. In a suitable embodiment the cancer is lymphoma, which may be selected from the group consisting of: Hodgkin's lymphoma; non-Hodgkin lymphoma; Burkitt's lymphoma; and small lymphocytic lymphoma.

In particular embodiments the cancer is selected from: breast cancer, esophageal cancer, bladder cancer, lung cancer, hematopoietic cancer, lymphoma, medulloblastoma, rectum adenocarcinoma, colon adenocarcinoma, gastric cancer, pancreatic cancer, liver cancer, adenoid cystic carcinoma, lung adenocarcinoma, head and neck squamous cell carcinoma, brain tumors, hepatocellular carcinoma, renal cell carcinoma, melanoma, oligodendroglioma, ovarian clear cell carcinoma, and ovarian serous.

In particular embodiments, the methods and uses disclosed herein provide a precision medicine approach, such as one that targets a particular type of tumour, or sub-set of patients with a particular tumour, or particular stage of tumour, or even an individual patient.

Suitably the methods of the first or second aspects of the invention serve to identify the cancer patient(s) most suited for treatment with a PRMT5 inhibitor. Suitably the treatment of such cancers may achieve effective treatment of the cancer by preventing or treating the development of the cancer, by preventing or treating the progression of the cancer, by preventing or treating the recurrence of the cancer, or by preventing or treating the propagation (including metastasis) of the cancer.

### Computer-Implemented Methods

According to a sixth aspect of the invention there is provided a computer-implemented method to aid in selecting a treatment for a patient with cancer, comprising the steps of:
(iv) receiving a value for the level of biomarker (a) pRb and/or (b) total E2F1 in a cancer cell containing biological sample from the patient;
(v) comparing the level(s) in (i) with a reference value for each biomarker,
(vi) wherein if the patient's cancer cells exhibit a decrease in level of pRb and/or an increase in level of total E2F1 compared to the reference value the patient is selected for treatment with a PRMT5 inhibitor.

Suitably, the level is expression level.

The term “computer-implemented” as used herein means that the method is carried out in an automated fashion on a data processing unit which is, typically, comprised in a computer or similar data processing device. The data processing unit shall receive values for the level of the biomarkers (i.e. pRb and/or (b) total E2F1). Such values can be the amounts, relative amounts or any other calculated value reflecting the amount as described elsewhere herein in detail. Accordingly, it is to be understood that the aforementioned method does not require the determination of amounts for the biomarkers but rather uses values for already predetermined amounts.

The present invention also, in principle, contemplates a computer program, computer program product or computer readable storage medium having tangibly embedded said computer program, wherein the computer program comprises instructions which, when run on a data processing device or computer, carry out the method of the present invention as specified above. Specifically, the present disclosure further encompasses:
a computer or computer network comprising at least one processor, wherein the processor is adapted to perform the method according to one of the aspects described in this description,
a computer loadable data structure that is adapted to perform the method according to one of the aspects described in this description while the data structure is being executed on a computer,
a computer script, wherein the computer program is adapted to perform the method according to one of the aspects described in this description while the program is being executed on a computer,
a computer program comprising program means for performing the method according to one of the aspects described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the aspects described in this description after having been loaded into a main and/or working storage of a computer or of a computer network,
a computer program product having program code means, wherein the program code means can be stored or are stored on a storage medium, for performing the method according to one of the aspects described in this description, if the program code means are executed on a computer or on a computer network,
a data stream signal, typically encrypted, comprising a data for parameters as defined herein elsewhere, and
a data stream signal, typically encrypted, comprising the assessment provided by the methods of the present invention.

### Kit

The present invention also includes kits, e.g., comprising one or more tools capable of quantifying the amount of pRb and/or E2F1 biomarkers in a sample.

According to a third aspect of the invention there is provided a kit for use in the method of the first aspect of the invention, which kit comprises one or more reagents capable of determining the expression level of pRb and/or total E2F1. Suitably, the kit comprises an antibody or antigen-binding portion thereof which specifically binds to E2F-1 protein; and/or an antibody or antigen-binding portion thereof which specifically binds to pRb protein; and/or a nucleic acid oligonucleotide capable of specifically binding to RB1 transcript; and/or a nucleic acid oligonucleotide capable of specifically binding to E2F1 transcript.

In particular embodiments, the oligonucleotide capable of specifically binding to one of the biomarker transcripts is a primer or a probe. Optionally, the primer or probe is labelled, such as with a fluorescent or radioactive label.

Typically, the kit will contain an antibody or antigen-binding moiety that is capable of binding to the biomarker in protein form (e.g. E2F1 protein or pRb protein) and/or a primer or probe capable of binding the biomarker in nucleic acid form (e.g. E2F1 transcript or RB1 transcript). Optionally such antibody or antigen-binding moiety, primer or probe may be labelled, such as fluorescently. The kit may also include instructions for use and may also contain additional elements needed to practice the method described on the instructions in the kit. The kit may also include information on interpreting the data and making a call, e.g. the threshold levels for interpreting whether the levels of the biomarker signify that the patient is likely to respond favourably to a PRMT5 inhibitor.

The kit comprises an antibody as described here, or epitope-binding fragment thereof. The kit may also comprise means for obtaining a biological sample, such as a spatula or a dipstick or a container for accepting the sample. The kit may also comprise one or more assay components for detecting the amount of total E2F-1 or pRb protein. Preferably the assay comprises an immunoassay, such as an ELISA. The kit may also comprise one or more assay components for detecting the amount of E2F-1 or RB1 transcript. Preferably the assay is RT-PCR.

In particular embodiments, the kit also comprises a positive control and/or a negative control. These process controls act as quality control to ensure valid assay results.

The presence and/or amount of total E2F-1 or pRB proteins in a sample may be determined by standard immunochemical techniques which are well known to the skilled person (for example immunohistochemistry, radioimmunoassay, ELISA, Western blot, fluorescence assay, DELFIA®, LANCE, FRET, etc). The method may preferably be carried out as a high throughput screen.

The presence and/or amount of E2F-1 or RB1 transcript in a sample may be determined by standard nucleic acid quantitation techniques which are well known to the skilled person (for example RT-PCR, qPCR).

An increase in the total E2F-1 protein or E2F-1 encoding transcript in a patient's cancer cell sample compared to a reference value, such as levels in a normal cell sample or normal reference value, indicates that the patient is suitable for treatment with a PRMT-5 inhibitor.

A decrease in the amount of pRb protein or RB1 transcript in a patient's cancer cell sample compared to the reference value (e.g. a normal cell sample or normal reference value) indicates that the patient is suitable for treatment with a PRMT-5 inhibitor.

Further aspects and embodiments will be apparent to those skilled in the art. All documents mentioned in this text are incorporated herein by reference. Aspects and embodiments of the present invention will now be discussed with reference to the following Examples and accompanying Figures.

### DESCRIPTION OF FIGURES

FIG. 1.

PRMT5 inhibition mediates cancer cell death and this effect is E2F1 dependent. U20S cells were transfected with PRMT5 (P5), E2F-1 (E2F1) or control (NC) or siRNA as indicated, and colony growth measured at 10 days after staining with crystal violet. (a) Levels of E2F1 and PRMT5 in siRNA treated cell is shown by western blot (b).

FIG. 2.

Cells with genetic deletion of E2F1 are less sensitive to PRMT5 inhibition.
(a) Cell viability assay in T-47D cell line (breast cancer) (b) Cell viability assay in HCT116 cell line (colorectal cancer).

FIG. 3.

Cells with genetic deletion of pRb are more sensitive to PRMT5 inhibition.
(a) Cell viability assay in T-47D cell line (breast cancer) (b) Cell viability assay in U20S cell line (bone cancer).

FIG. 4.

Cells with genetic deletion of E2F1 are less sensitive to PRMT5 inhibitor GSK3326595.
(a) Cell viability assay in HCT116 cell line (colorectal cancer) (b) Cell viability assay in T-47D cell line (breast cancer).

FIG. 5.

Cells with genetic deletion of E2F1 are less sensitive to PRMT5 inhibitor PF06939999.
(a) Cell viability assay in HCT116 cell line (colorectal cancer) (b) Cell viability assay in T-47D cell line (breast cancer).

FIG. 6.

Cell viability assay in HCT116 cell line (colorectal cancer).

Cells with genetic deletion of E2F1 are less sensitive to PRMT5 inhibitor JNJ64619178.

FIG. 7.

Cell viability assay in HCT116 cell line (colorectal cancer).

Cells with genetic deletion of E2F1 are less sensitive to PRMT5 inhibitor LLY283.

FIG. 8.

Cell viability assay in T-47D cell line (breast cancer). Cells with genetic deletion of pRb are more sensitive to PRMT5 inhibitor GSK3326595.

FIG. 9.

Cell viability assay in T-47D cell line (breast cancer). Cells with genetic deletion of pRb are more sensitive to PRMT5 inhibitor PF06939999.

Sequences:
Gene Name: RB1 (RB transcriptional corepressor 1)
Gene ID: 5925
Protein ID: NP_000312.2
CCDS: CCDS31973.1
RB1 Nucleotide Sequence (2787 nt):
SEQ ID NO: 1
ATGCCGCCCAAAACCCCCCGAAAAACGGCCGCCACCGCCGCCGCTGCCGC
CGCGGAACCCCCGGCACCGCCGCCGCCGCCCCCTCCTGAGGAGGACCCAG
AGCAGGACAGCGGCCCGGAGGACCTGCCTCTCGTCAGGCTTGAGTTTGAA
GAAACAGAAGAACCTGATTTTACTGCATTATGTCAGAAATTAAAGATACC
AGATCATGTCAGAGAGAGAGCTTGGTTAACTTGGGAGAAAGTTTCATCTG
TGGATGGAGTATTGGGAGGTTATATTCAAAAGAAAAAGGAACTGTGGGGA
ATCTGTATCTTTATTGCAGCAGTTGACCTAGATGAGATGTCGTTCACTTT
TACTGAGCTACAGAAAAACATAGAAATCAGTGTCCATAAATTCTTTAACT
TACTAAAAGAAATTGATACCAGTACCAAAGTTGATAATGCTATGTCAAGA
CTGTTGAAGAAGTATGATGTATTGTTTGCACTCTTCAGCAAATTGGAAAG
GACATGTGAACTTATATATTTGACACAACCCAGCAGTTCGATATCTACTG
AAATAAATTCTGCATTGGTGCTAAAAGTTTCTTGGATCACATTTTTATTA
GCTAAAGGGGAAGTATTACAAATGGAAGATGATCTGGTGATTTCATTTCA
GTTAATGCTATGTGTCCTTGACTATTTTATTAAACTCTCACCTCCCATGT
TGCTCAAAGAACCATATAAAACAGCTGTTATACCCATTAATGGTTCACCT
CGAACACCCAGGCGAGGTCAGAACAGGAGTGCACGGATAGCAAAACAACT
AGAAAATGATACAAGAATTATTGAAGTTCTCTGTAAAGAACATGAATGTA
ATATAGATGAGGTGAAAAATGTTTATTTCAAAAATTTTATACCTTTTATG
AATTCTCTTGGACTTGTAACATCTAATGGACTTCCAGAGGTTGAAAATCT
TTCTAAACGATACGAAGAAATTTATCTTAAAAATAAAGATCTAGATGCAA
GATTATTTTTGGATCATGATAAAACTCTTCAGACTGATTCTATAGACAGT
TTTGAAACACAGAGAACACCACGAAAAAGTAACCTTGATGAAGAGGTGAA
TGTAATTCCTCCACACACTCCAGTTAGGACTGTTATGAACACTATCCAAC
AATTAATGATGATTTTAAATTCAGCAAGTGATCAACCTTCAGAAAATCTG
ATTTCCTATTTTAACAACTGCACAGTGAATCCAAAAGAAAGTATACTGAA
AAGAGTGAAGGATATAGGATACATCTTTAAAGAGAAATTTGCTAAAGCTG
TGGGACAGGGTTGTGTCGAAATTGGATCACAGCGATACAAACTTGGAGTT
CGCTTGTATTACCGAGTAATGGAATCCATGCTTAAATCAGAAGAAGAACG
ATTATCCATTCAAAATTTTAGCAAACTTCTGAATGACAACATTTTTCATA
TGTCTTTATTGGCGTGCGCTCTTGAGGTTGTAATGGCCACATATAGCAGA
AGTACATCTCAGAATCTTGATTCTGGAACAGATTTGTCTTTCCCATGGAT
TCTGAATGTGCTTAATTTAAAAGCCTTTGATTTTTACAAAGTGATCGAAA
GTTTTATCAAAGCAGAAGGCAACTTGACAAGAGAAATGATAAAACATTTA
GAACGATGTGAACATCGAATCATGGAATCCCTTGCATGGCTCTCAGATTC
ACCTTTATTTGATCTTATTAAACAATCAAAGGACCGAGAAGGACCAACTG
ATCACCTTGAATCTGCTTGTCCTCTTAATCTTCCTCTCCAGAATAATCAC
ACTGCAGCAGATATGTATOTTTCTCCTGTAAGATCTCCAAAGAAAAAAGG
TTCAACTACGCGTGTAAATTCTACTGCAAATGCAGAGACACAAGCAACCT
CAGCCTTCCAGACCCAGAAGCCATTGAAATCTACCTCTCTTTCACTGTTT
TATAAAAAAGTGTATCGGCTAGCCTATCTCCGGCTAAATACACTTTGTGA
ACGCCTTCTGTCTGAGCACCCAGAATTAGAACATATCATCTGGACCCTTT
TCCAGCACACCCTGCAGAATGAGTATGAACTCATGAGAGACAGGCATTTG
GACCAAATTATGATGTGTTCCATGTATGGCATATGCAAAGTGAAGAATAT
AGACCTTAAATTCAAAATCATTGTAACAGCATACAAGGATCTTCCTCATG
CTGTTCAGGAGACATTCAAACGTGTTTTGATCAAAGAAGAGGAGTATGAT
TCTATTATAGTATTCTATAACTCGGTCTTCATGCAGAGACTGAAAACAAA
TATTTTGCAGTATGCTTCCACCAGGCCCCCTACCTTGTCACCAATACCTC
ACATTCCTCGAAGCCCTTACAAGTTTCCTAGTTCACCCTTACGGATTCCT
GGAGGGAACATCTATATTTCACCCCTGAAGAGTCCATATAAAATTTCAGA
AGGTCTGCCAACACCAACAAAAATGACTCCAAGATCAAGAATCTTAGTAT
CAATTGGTGAATCATTCGGGACTTCTGAGAAGTTCCAGAAAATAAATCAG
ATGGTATGTAACAGCGACCGTGTGCTCAAAAGAAGTGCTGAAGGAAGCAA
CCCTCCTAAACCACTGAAAAAACTACGCTTTGATATTGAAGGATCAGATG
AAGCAGATGGAAGTAAACATCTCCCAGGAGAGTCCAAATTTCAGCAGAAA
CTGGCAGAAATGACTTCTACTCGAACACGAATGCAAAAGCAGAAAATGAA
TGATAGCATGGATACCTCAAACAAGGAAGAGAAATGA
Translation (928 aa):
SEQ ID NO: 2
MPPKTPRKTAATAAAAAAEPPAPPPPPPPEEDPEQDSGPEDLPLVRLEFE
ETEEPDFTALCQKLKIPDHVRERAWLTWEKVSSVDGVLGGYIQKKKELWG
ICIFIAAVDLDEMSFTFTELQKNIEISVHKFFNLLKEIDTSTKVDNAMSR
LLKKYDVLFALFSKLERTCELIYLTQPSSSISTEINSALVLKVSWITFLL
AKGEVLQMEDDLVISFQLMLCVLDYFIKLSPPMLLKEPYKTAVIPINGSP
RTPRRGQNRSARIAKQLENDTRIIEVLCKEHECNIDEVKNVYFKNFIPFM
NSLGLVTSNGLPEVENLSKRYEEIYLKNKDLDARLFLDHDKTLQTDSIDS
FETQRTPRKSNLDEEVNVIPPHTPVRTVMNTIQQLMMILNSASDQPSENL
ISYFNNCTVNPKESILKRVKDIGYIFKEKFAKAVGQGCVEIGSQRYKLGV
RLYYRVMESMLKSEEERLSIQNFSKLLNDNIFHMSLLACALEVVMATYSR
STSQNLDSGTDLSFPWILNVLNLKAFDFYKVIESFIKAEGNLTREMIKHL
ERCEHRIMESLAWLSDSPLFDLIKQSKDREGPTDHLESACPLNLPLQNNH
TAADMYLSPVRSPKKKGSTTRVNSTANAETQATSAFQTQKPLKSTSLSLF
YKKVYRLAYLRLNTLCERLLSEHPELEHIIWTLFQHTLQNEYELMRDRHL
DQIMMCSMYGICKVKNIDLKFKIIVTAYKDLPHAVQETFKRVLIKEEEYD
SIIVFYNSVFMQRLKTNILQYASTRPPTLSPIPHIPRSPYKFPSSPLRIP
GGNIYISPLKSPYKISEGLPTPTKMTPRSRILVSIGESFGTSEKFQKINQ
MVCNSDRVLKRSAEGSNPPKPLKKLRFDIEGSDEADGSKHLPGESKFQQK
LAEMTSTRTRMQKQKMNDSMDTSNKEEK
Gene Name: E2F1 (E2F transcription factor 1)
Gene ID: 1869
Protein ID: NP_005216-1
CCDS: CCDS13224.1
E2F1 Nucleotide Sequence (1314 nt):
SEQ ID NO: 3
ATGGCCTTGGCCGGGGCCCCTGCGGGCGGCCCATGCGCGCCGGCGCTGGA
GGCCCTGCTCGGGGCCGGCGCGCTGCGGCTGCTCGACTCCTCGCAGATCG
TCATCATCTCCGCCGCGCAGGACGCCAGCGCCCCGCCGGCTCCCACCGGC
CCCGCGGCGCCCGCCGCCGGCCCCTGCGACCCTGACCTGCTGCTCTTCGC
CACACCGCAGGCGCCCCGGCCCACACCCAGTGCGCCGCGGCCCGCGCTCG
GCCGCCCGCCGGTGAAGCGGAGGCTGGACCTGGAAACTGACCATCAGTAC
CTGGCCGAGAGCAGTGGGCCAGCTCGGGGCAGAGGCCGCCATCCAGGAAA
AGGTGTGAAATCCCCGGGGGAGAAGTCACGCTATGAGACCTCACTGAATC
TGACCACCAAGCGCTTCCTGGAGCTGCTGAGCCACTCGGCTGACGGTGTC
GTCGACCTGAACTGGGCTGCCGAGGTGCTGAAGGTGCAGAAGCGGCGCAT
CTATGACATCACCAACGTCCTTGAGGGCATCCAGCTCATTGCCAAGAAGT
CCAAGAACCACATCCAGTGGCTGGGCAGCCACACCACAGTGGGCGTCGGC
GGACGGCTTGAGGGGTTGACCCAGGACCTCCGACAGCTGCAGGAGAGCGA
GCAGCAGCTGGACCACCTGATGAATATCTGTACTACGCAGCTGCGCCTGC
TCTCCGAGGACACTGACAGCCAGCGCCTGGCCTACGTGACGTGTCAGGAC
CTTCGTAGCATTGCAGACCCTGCAGAGCAGATGGTTATGGTGATCAAAGC
CCCTCCTGAGACCCAGCTCCAAGCCGTGGACTCTTCGGAGAACTTTCAGA
TCTCCCTTAAGAGCAAACAAGGCCCGATCGATGTTTTCCTGTGCCCTGAG
GAGACCGTAGGTGGGATCAGCCCTGGGAAGACCCCATCCCAGGAGGTCAC
TTCTGAGGAGGAGAACAGGGCCACTGACTCTGCCACCATAGTGTCACCAC
CACCATCATCTCCCCCCTCATCCCTCACCACAGATCCCAGCCAGTCTCTA
CTCAGCCTGGAGCAAGAACCGCTGTTGTCCCGGATGGGCAGCCTGGGGCT
CCCGTGGACGAGGACCGCCTGTCCCCGCTGGTGGCGGCCGACTCGCTCCT
GGAGCATGTGCGGGAGGACTTCTCCGGCCTCCTCCCTGAGGAGTTCATCA
GCCTTTCCCCACCCCACGAGGCCCTCGACTACCACTTCGGCCTCGAGGAG
GGCGAGGGCATCAGAGACCTCTTCGACTGTGACTTTGGGGACCTCACCCC
CCTGGATTTCTGA
Translation (437 aa):
SEQ ID NO: 4
MALAGAPAGGPCAPALEALLGAGALRLLDSSQIVIISAAQDASAPPAPTG
PAAPAAGPCDPDLLLFATPQAPRPTPSAPRPALGRPPVKRRLDLETDHQY
LAESSGPARGRGRHPGKGVKSPGEKSRYETSLNLTTKRFLELLSHSADGV
VDLNWAAEVLKVQKRRIYDITNVLEGIQLIAKKSKNHIQWLGSHTTVGVG
GRLEGLTQDLRQLQESEQQLDHLMNICTTQLRLLSEDTDSQRLAYVTCQD
LRSIADPAEQMVMVIKAPPETQLQAVDSSENFQISLKSKQGPIDVFLCPE
ETVGGISPGKTPSQEVTSEEENRATDSATIVSPPPSSPPSSLTTDPSQSL
LSLEQEPLLSRMGSLRAPVDEDRLSPLVAADSLLEHVREDFSGLLPEEFI
SLSPPHEALDYHFGLEEGEGIRDLFDCDFGDLTPLDF
PRMT5 siRNA sequence
SEQ ID NO: 5
CCG CUA UUG CAC CUU GGA A
E2F-1 siRNA sequence:
SEQ ID NO: 6
AAC UCC UCG CAG AUC GUC AUC

### EXAMPLES

### Example 1

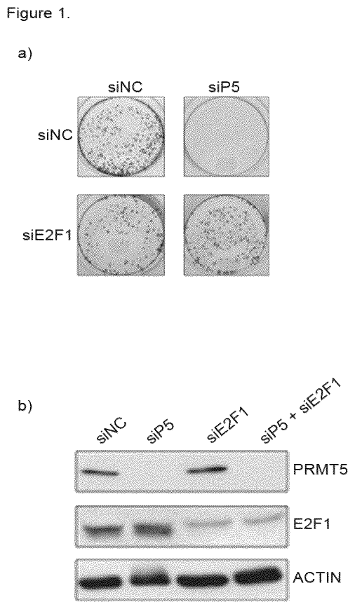

The effect of PRMT5 and E2F-1 in growth control was evaluated in U20S cells. When an analysis of cell growth was performed, the inhibitory effect of PRMT5 was evident when cell growth was measured in the context of a colony formation assay, where PRMT5 siRNA caused a dramatic reduction in growth after 10 days, which was rescued upon co-depletion of E2F-1 (FIG. 1a). Protein levels of PRMT5 and E2F1 are shown in FIG. 1b. Silencing of PRMT5 activity, with gene-specific siRNA, results in cell death through apoptosis. Moreover, this cell death requires E2F1 activity, as co-silencing E2F1 and PRMT5 rescues the cell death and allows cells to continue growing (FIG. 1a).

### Example 2

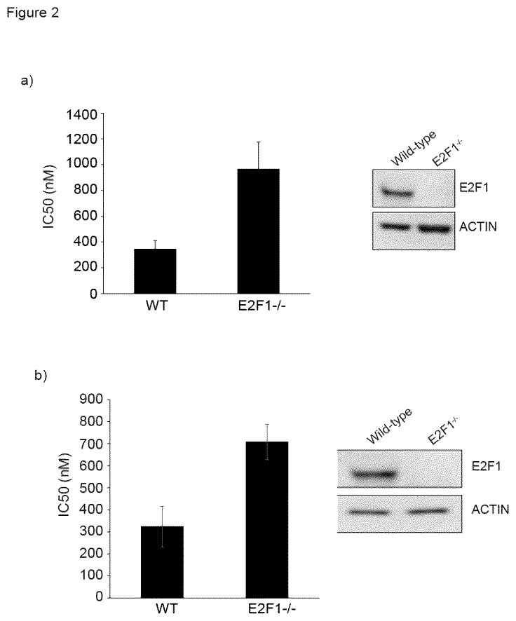

Tumour cells that lack the E2F1 gene are less sensitive to PRMT5 inhibition mediated by AT101. In colorectal cancer and breast cancer cell lines where E2F1 has been deleted using CRISPR technology, depletion of E2F1 desensitises cells to PRMT5 inhibition (FIGS. 2 (a) and (b), indicating that E2F1 is a positive measure for response to PRMT5 inhibition.

### Example 3

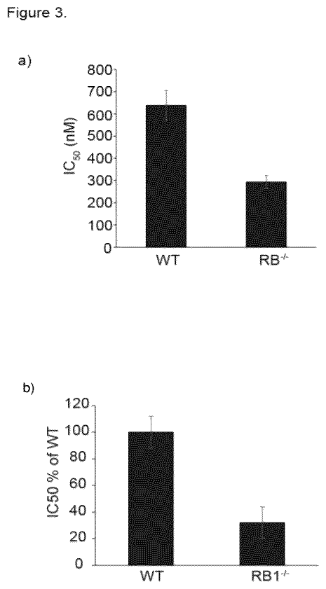

Tumour cells that lack the tumour supressor protein pRb gene are more sensitive to PRMT5 inhibition mediated by AT101. In breast cancer and bone cancer cell lines where pRb has been deleted using CRISPR technology, depletion of pRb sensitises cells to PRMT5 inhibition (FIGS. 3 (a) and (b), indicating that pRb is a negative measure for response to PRMT5 inhibition.

### Example 4

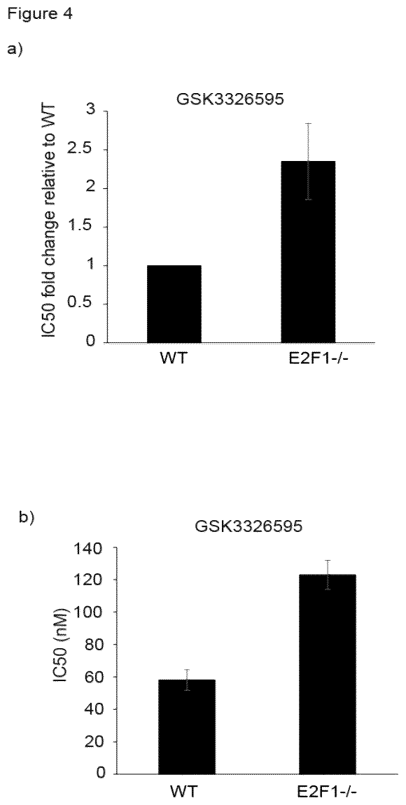

Tumour cells that lack the E2F1 gene are less sensitive to PRMT5 inhibition mediated by GSK3326595. In colorectal cancer and breast cancer cell lines where E2F1 has been deleted using CRISPR technology, depletion of E2F1 desensitises cells to GSK3326595 PRMT5 inhibition (FIGS. 4 (a) and (b)), indicating that E2F1 is a positive measure for response to GSK3326595 mediated PRMT5 inhibition.

### Example 5

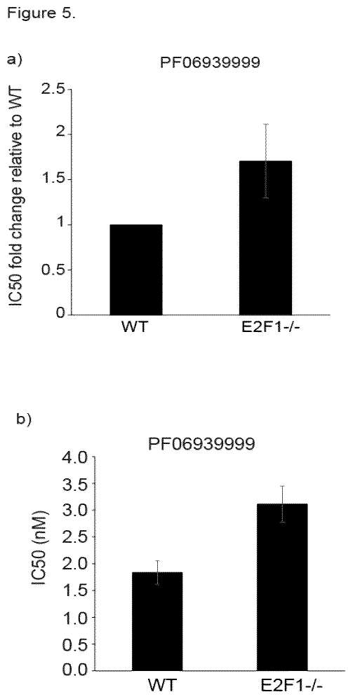

Tumour cells that lack the E2F1 gene are less sensitive to PRMT5 inhibition mediated by PF06939999. In colorectal cancer and breast cancer cell lines where E2F1 has been deleted using CRISPR technology, depletion of E2F1 desensitises cells to PF06939999 PRMT5 inhibition (FIGS. 5 (a) and (b)), indicating that E2F1 is a positive measure for response to PF06939999 mediated PRMT5 inhibition.

### Example 6

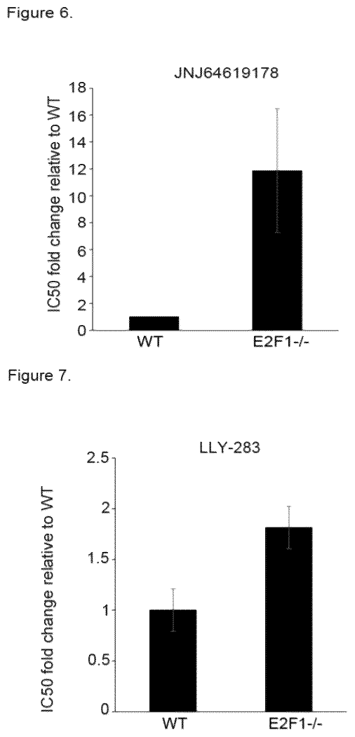

Tumour cells that lack the E2F1 gene are less sensitive to PRMT5 inhibition mediated by JNJ64619178. In the breast cancer cell line T-47D where E2F1 has been deleted using CRISPR technology, depletion of E2F1 desensitises cells to JNJ64619178 PRMT5 inhibition (FIG. 6) indicating that E2F1 is a positive measure for response to JNJ64619178 mediated PRMT5 inhibition.

### Example 7

Tumour cells that lack the E2F1 gene are less sensitive to PRMT5 inhibition mediated by LLY-283. In the breast cancer cell line T-47D where E2F1 has been deleted using CRISPR technology, depletion of E2F1 desensitises cells to LLY-283 PRMT5 inhibition (FIG. 7) indicating that E2F1 is a positive measure for response to LLY-283 mediated PRMT5 inhibition.

### Example 8

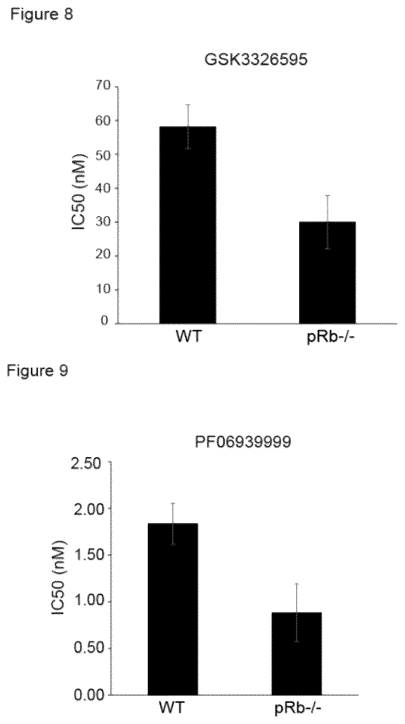

Tumour cells that lack the tumour supressor protein pRb gene are more sensitive to PRMT5 inhibition mediated by GSK3326595. In breast cancer cancer cell lines where pRb has been deleted using CRISPR technology, depletion of pRb sensitises cells to GSK3326595 PRMT5 inhibition (FIG. 8), indicating that pRb is a negative measure for response to GSK3326595 mediated PRMT5 inhibition.

### Example 9

Tumour cells that lack the tumour supressor protein pRb gene are more sensitive to PRMT5 inhibition mediated by PF06939999. In breast cancer cancer cell lines where pRb has been deleted using CRISPR technology, depletion of pRb sensitises cells to PF06939999 PRMT5 inhibition (FIG. 9), indicating that pRb is a negative measure for response to PF06939999 mediated PRMT5 inhibition.

### Material and Methods

### MT Assay

T-47D cells were maintained in growth medium (RPMI 1640 supplemented with 10% v/v heat inactivated fetal bovine serum and cultured at 37° C., 5% CO₂, U2OS, MCF7 and HCT116 cells were maintained in growth medium (DMEM supplemented with 10% v/v heat inactivated fetal bovine serum) and cultured at 37° C., 5% CO₂. Under assay conditions, cells were incubated in assay medium (RPMI 1640 or DMSO supplemented with 10% v/v heat inactivated fetal bovine serum and 100 units/mL penicillin-streptomycin) at 37° C. under 5% CO₂. For the assessment of the effect of compounds on the proliferation of the cancer cell lines, exponentially growing cells were plated into 96-well plates overnight at a density of 1,000 cells/well in a final volume of 100 μl of cell growth medium. The next day the cells were dosed with compounds (T-992, GSK3326595, LLY-283, PF-06939999 and JNJ64619178) (quadruplicate ten-point 5-fold serial dilutions in DMSO), beginning at 100 μM. After addition of compounds, assay plates were incubated for 8 days at 37° C., 5% CO₂, relative humidity 90%. NAD(P)H-dependent cellular oxidoreductase enzyme activity was measured by adding 100 μl thiazolyl blue tetrazolium bromide (MTT: Sigma-Aldrich) to the wells at a final concentration of 5 μM and incubated for 2 h at 37° C. Next, media was discarded from the wells and the formazan crystals were dissolved in 100 μl DMSO by shaking for 15 min. Absorbance was read on the Omega FLUOstar plate reader (BMG Labtech Ltd, Ortenberg, Germany) at a wavelength of 584 nM. Data were analysed and IC₅₀ values determined using MARs data analysis software (BMG Labtech Ltd, Ortenberg, Germany). The concentration of compound inhibiting cell viability by 50% was determined using a 4-parametric fit of the normalized dose response curves.

### Western Blot

Cells were harvested, washed in PBS, and resuspended in lysis buffer [50 mM Tris pH 7.4, 5 mM EDTA, 0.5% Igepal CA-630 (Sigma, Gillingham, UK), 50 mM NaF, 1 mM DTT, 0.2 mM Na₃VO₄, 120 mM NaCl, protease inhibitor cocktail]. Total protein concentration was determined by Bradford Assay (Bio-Rad). Whole cell lysates were prepared and lysates were separated by 4-20% SDS-PAGE (Bio-rad) and transferred to PVDF membrane (GE Healthcare, Piscataway, NJ). Membranes were blocked with 5% milk in PBS with 0.2% Tween 20 for 1 h at room temperature. Primary antibodies for actin (Sigma, A2228), E2F1 (CST #3742) and Rb (#9309) were diluted 1:1000 in 5% milk incubated overnight at 4° C. Membranes were washed with PBS-T, incubated with HRP-conjugated secondary antibodies (CST) diluted in PBS-T with 5% milk for 1 h at room temperature, washed, and developed using SuperSignal West Dura Chemiluminescent Substrate (Thermo Scientific).

### Colony Formation Assay

U2OS cells were seeded at a density of 1000 cells/well in a 6-well plate and transfection of siRNA was performed as described in the section below. U2OS cells were allowed to establish colonies over a period of 10 days before ending the experiment. The culturing media was gently aspirated to avoid physically damaging the cells and the plates were briefly rinsed with PBS. Crystal violet (Sigma-Aldrich) stain (0.5%) was applied to the cells for 2 minutes, followed by rinsing with autoclaved deionised water and left to air dry. Plates were scanned and colonies measured and counted using the Gelcount™ Colony Counter (Oxford Optronics).

siRNA Transfection

Oligofectamine reagent (Invitrogen) and siRNA complexes (non-targeting, E2F1 and PRMT5) were incubated separately with OPTI-MEM® I Reduced Serum Media (Gibco®) for 5 minutes at room temperature. The two mixtures were combined and incubated for a further 20 minutes. siRNA transfection mix was added to U20S cells in a dropwise fashion. Commercial non-targeting siRNA control was from Dharmacon. PRMT5 siRNA sequence: 5′-CCG CUA UUG CAC CUU GGA A-3′ (SEQ ID NO: 5), E2F-1 siRNA sequence: 5′-AAC UCC UCG CAG AUC GUC AUC-3′ (SEQ ID NO: 6) [sense strands shown]. In experiments involving treatment of more than one siRNA, non-targeting siRNA was included to ensure equal amounts of transfected siRNA across all samples.

### CRISPR-Cas9 Gene Disruption

pSpCas9(BB)-2A-Puro (Addgene plasmid ID: 48139) was used for the generation of E2F1 and pRb knock out cell lines using CRISPR-Cas9 technology. Cell lines were generated as detailed in (Ran et al., 2013).

### REFERENCES

Bandara, L. R., & La Thangue, N. B. (1991). Adenovirus E1a prevents the retinoblastoma gene product from complexing with a cellular transcription factor. Nature, 351(6326), 494-497. https://doi.org/10.1038/351494a0
Barczak, W., Jin, L., Carr, S. M., Munro, S., Ward, S., Kanapin, A., Samsonova, A., & La Thangue, N. B. (2020). PRMT5 promotes cancer cell migration and invasion through the E2F pathway. Cell Death & Disease, 11(7), 572. https://doi.org/10.1038/s41419-020-02771-9 Bedford, M. T., & Clarke, S. G. (2009). Protein arginine methylation in mammals: Who, what, and why. Molecular Cell, 33(1), 1-13. https://doi.org/10.1016/j.molcel.2008.12.013
Benedict, W. F., Lerner, S. P., Zhou, J., Shen, X., Tokunaga, H., & Czerniak, B. (1999). Level of retinoblastoma protein expression correlates with p16 (MTS-1/INK4A/CDKN2) status in bladder cancer. Oncogene, 18(5), 1197-1203. https://doi.org/10.1038/sj.onc.1202452
Bhateja, P., Chiu, M., Wildey, G., Lipka, M. B., Fu, P., Yang, M. C. L., Ardeshir-Larijani, F., Sharma, N., & Dowlati, A. (2019). Retinoblastoma mutation predicts poor outcomes in advanced non small cell lung cancer. Cancer Medicine, 8(4), 1459-1466. https://doi.org/10.1002/cam4.2023
Cho, E.-C., Zheng, S., Munro, S., Liu, G., Carr, S. M., Moehlenbrink, J., Lu, Y.-C., Stimson, L., Khan, O., Konietzny, R., McGouran, J., Coutts, A. S., Kessler, B., Kerr, D. J., & Thangue, N. B. L. (2012). Arginine methylation controls growth regulation by E2F-1. The EMBO Journal, 31(7), 1785-1797. https://doi.org/10.1038/emboj.2012.17Ferreira de Freitas, R., Ivanochko, D., and Schapira, M. (2019). Methyltransferase Inhibitors: Competing with, or Exploiting the Bound Cofactor. Molecules 24, 4492.
Derenzini, M., Donati, G., Mazzini, G., Montanaro, L., Vici, M., Ceccarelli, C., Santini, D., Taffurelli, M., & Treré, D. (2008). Loss of retinoblastoma tumor suppressor protein makes human breast cancer cells more sensitive to antimetabolite exposure. Clinical Cancer Research: An Official Journal of the American Association for Cancer Research, 14(7), 2199-2209. https://doi.org/10.1158/1078-0432.CCR-07-2065
Dick, F. A., & Rubin, S. M. (2013). Molecular mechanisms underlying RB protein function. Nature Reviews. Molecular Cell Biology, 14(5), 297-306. https://doi.org/10.1038/nrm3567
Ertel, A., Dean, J. L., Rui, H., Liu, C., Witkiewicz, A. K., Knudsen, K. E., & Knudsen, E. S. (2010). RB-pathway disruption in breast cancer. Cell Cycle, 9(20), 4153-4163. https://doi.org/10.4161/cc.9.20.13454
Field, S. J., Tsai, F. Y., Kuo, F., Zubiaga, A. M., Kaelin, W. G., Livingston, D. M., Orkin, S. H., & Greenberg, M. E. (1996). E2F-1 functions in mice to promote apoptosis and suppress proliferation. Cell, 85(4), 549-561. https://doi.org/10.1016/s0092-8674(00)81255-6
Frolov, M. V., & Dyson, N. J. (2004). Molecular mechanisms of E2F-dependent activation and pRB-mediated repression. Journal of Cell Science, 117(Pt 11), 2173-2181. https://doi.org/10.1242/jcs.01227
Hanahan, D., & Weinberg, R. A. (2000). The hallmarks of cancer. Cell, 100(1), 57-70. https://doi.org/10.1016/s0092-8674(00)81683-9
Iaquinta, P. J., & Lees, J. A. (2007). Life and death decisions by the E2F transcription factors. Current Opinion in Cell Biology, 19(6), 649-657. https://doi.org/10.1016/j.ceb.2007.10.006
Jackson, S. P., & Bartek, J. (2009). The DNA-damage response in human biology and disease. Nature, 461(7267), 1071-1078. https://doi.org/10.1038/nature08467
Jarrold, J., & Davies, C. C. (2019). PRMTs and Arginine Methylation: Cancer's Best-Kept Secret?Trends in Molecular Medicine, 25(11), 993-1009. https://doi.org/10.1016/j.molmed.2019.05.007
Kim, H., & Ronai, Z. A. (2020). PRMT5 function and targeting in cancer. Cell Stress, 4(8), 199-215. https://doi.org/10.15698/cst2020.08.228
Knudsen, E. S., Nambiar, R., Rosario, S. R., Smiraglia, D. J., Goodrich, D. W., & Witkiewicz, A. K. (2020). Pan-cancer molecular analysis of the RB tumor suppressor pathway. Communications Biology, 3(1), 1-12. https://doi.org/10.1038/s42003-020-0873-9
Lacombe, C., De Rycke, O., Couvelard, A., Turpin, A., Cazes, A., Hentic, O., Gounant, V., Zalcman, G., Ruszniewski, P., Cros, J., & de Mestier, L. (2021). Biomarkers of Response to Etoposide-Platinum Chemotherapy in Patients with Grade 3 Neuroendocrine Neoplasms. Cancers, 13(4), 643. https://doi.org/10.3390/cancers13040643
Pediconi, N., Ianari, A., Costanzo, A., Belloni, L., Gallo, R., Cimino, L., Porcellini, A., Screpanti, I., Balsano, C., Alesse, E., Gulino, A., & Levrero, M. (2003). Differential regulation of E2F1 apoptotic target genes in response to DNA damage. Nature Cell Biology, 5(6), 552-558. https://doi.org/10.1038/ncb998
Polager, S., & Ginsberg, D. (2008). E2F—at the crossroads of life and death. Trends in Cell Biology, 18(11), 528-535. https://doi.org/10.1016/.tcb.2008.08.003
Ran, F. A., Hsu, P. D., Wright, J., Agarwala, V., Scott, D. A., & Zhang, F. (2013). Genome engineering using the CRISPR-Cas9 system. Nature Protocols, 8(11), 2281-2308. https://doi.org/10.1038/nprot.2013.143
Shailesh, H., Zakaria, Z. Z., Baiocchi, R., & Sif, S. (2018). Protein arginine methyltransferase 5 (PRMT5) dysregulation in cancer. Oncotarget, 9(94), 36705-36718. https://doi.org/10.18632/oncotarget.26404
Stevens, C., & La Thangue, N. B. (2003). E2F and cell cycle control: A double-edged sword. Archives of Biochemistry and Biophysics, 412(2), 157-169. https://doi.org/10.1016/s0003-9861(03)00054-7
Stevens, C., Smith, L., & La Thangue, N. B. (2003). Chk2 activates E2F-1 in response to DNA damage. Nature Cell Biology, 5(5), 401-409. https://doi.org/10.1038/ncb974
Tsai, K. Y., Hu, Y., Macleod, K. F., Crowley, D., Yamasaki, L., & Jacks, T. (1998). Mutation of E2f-1 suppresses apoptosis and inappropriate S phase entry and extends survival of Rb-deficient mouse embryos. Molecular Cell, 2(3), 293-304. https://doi.org/10.1016/s1097-2765(00)80274-9
van den Heuvel, S., & Dyson, N. J. (2008). Conserved functions of the pRB and E2F families. Nature Reviews. Molecular Cell Biology, 9(9), 713-724. https://doi.org/10.1038/nrm2469
Xing, E. P., Yang, G. Y., Wang, L. D., Shi, S. T., & Yang, C. S. (1999). Loss of heterozygosity of the Rb gene correlates with pRb protein expression and associates with p53 alteration in human esophageal cancer. Clinical Cancer Research: An Official Journal of the American Association for Cancer Research, 5(5), 1231-1240.
Zamanian, M., & La Thangue, N. B. (1992). Adenovirus E1a prevents the retinoblastoma gene product from repressing the activity of a cellular transcription factor. The EMBO Journal, 11(7), 2603-2610.
Zheng, S., Moehlenbrink, J., Lu, Y.-C., Zalmas, L.-P., Sagum, C. A., Carr, S., McGouran, J. F., Alexander, L., Fedorov, O., Munro, S., Kessler, B., Bedford, M. T.,
Yu, Q., & La Thangue, N. B. (2013). Arginine methylation-dependent reader-writer interplay governs growth control by E2F-1. Molecular Cell, 52(1), 37-51. https://doi.org/10.1016/j.molcel.2013.08.03
Yu, Q., & La Thangue, N. B. (2013). Arginine methylation-dependent reader-writer interplay governs growth control by E2F-1. Molecular Cell, 52(1), 37-51. https://doi.org/10.1016/j.molcel.2013.08.039

1. A method of selecting a treatment for a patient with cancer comprising:
(i) determining the expression level of biomarker (a) pRb and/or (b) total E2F1 in a cancer cell containing biological sample from the patient;
(ii) comparing the expression level(s) in (i) with a reference value for each biomarker,
(iii) wherein if the patient's cancer cells exhibit a decrease in expression of pRb and/or an increase in expression of total E2F1 compared to the reference value the patient is selected for treatment with a PRMT5 inhibitor.

2. The method according to claim, wherein the expression level is expression of protein or nucleic acid.

3. The method of claim 1 or 2, wherein the determination is based on expression level of total E2F1 protein.

4. The method of claim 1 or 2, wherein the determination is based on expression level of pRb protein.

5. The method of claim 1 or 2, wherein the determination is based on expression level of pRb and total E2F1.

6. The method of any one of the preceding claims, wherein the level of protein is detected using an antibody that binds to the protein.

7. The method of claim 6, wherein the antibody is a monoclonal antibody.

8. The method according to any one of claims 1, 2, 4 and 5, wherein reduced expression level of pRb is determined indirectly by identification of a RB1 mutation indicative of reduced or null expression in the cancer cells.

9. A method of selecting a treatment for a patient with cancer comprising determining whether the PRB1 gene in the cancer cells comprises one or more mutations that result in reduced, including null, expression of pRb, wherein if the cancer cells comprise one or more mutations in PRB1 gene that result in reduced expression of pRb the patient is selected for treatment with a PRMT5 inhibitor.

10. The method according to any one of the preceding claims wherein the cancer is selected from selected from the group consisting of: leukaemia, lymphoma, multiple myeloma, lung cancer, liver cancer, breast cancer, head and neck cancer, neuroblastoma, thyroid carcinoma, skin cancer (including melanoma), oral squamous cell carcinoma, urinary bladder cancer, Leydig cell tumour, biliary cancer, such as cholangiocarcinoma or bile duct cancer, brain cancer, pancreatic cancer, colon cancer, colorectal cancer and gynaecological cancers, including ovarian cancer, endometrial cancer, fallopian tube cancer, uterine cancer and cervical cancer, including epithelia cervix carcinoma. In suitable embodiments, the cancer is leukaemia and can be selected from the group consisting of acute lymphoblastic leukaemia, acute myelogenous leukaemia (also known as acute myeloid leukaemia or acute non-lymphocytic leukaemia), acute promyelocytic leukaemia, acute lymphocytic leukaemia, chronic myelogenous leukaemia (also known as chronic myeloid leukaemia, chronic myelocytic leukaemia or chronic granulocytic leukaemia), chronic lymphocytic leukaemia, monoblastic leukaemia and hairy cell leukaemia. In further preferred embodiments, the cancer is acute lymphoblastic leukaemia. In a suitable embodiment the cancer is lymphoma, which may be selected from the group consisting of: Hodgkin's lymphoma; non-Hodgkin lymphoma; Burkitt's lymphoma; and small lymphocytic lymphoma.

11. A kit for use in a method of identifying cancer which may be susceptible to treatment by the inhibition of PRMT5, wherein the kit comprises an antibody or antigen-binding portion thereof which specifically binds to E2F-1 protein; and/or an antibody or antigen-binding portion thereof which specifically binds to pRb protein; and/or a nucleic acid oligonucleotide capable of specifically binding to RB1 transcript; and/or a nucleic acid oligonucleotide capable of specifically binding to E2F1 transcript.

12. A kit for use according to claim 11, wherein the kit also comprises assay reagents for detection of antibody target binding or nucleic acid oligonucleotide target binding and/or instructions for use.

13. A PRMT5 inhibitor for use in treating a cancer whose cells express greater than normal levels of total E2F1 protein and/or reduced levels of pRb protein compared to normal.

14. The PRMT5 inhibitor for use according to claim 13, wherein the cancer cells express greater than normal levels of total E2F1 protein.

15. The PRMT5 inhibitor for use according to claim 13 or 14, wherein the cancer cells express reduced levels of pRb protein compared to normal.

16. The PRMT5 inhibitor for use according to any one of claims 13 to 15, wherein the cancer cells express greater than normal levels of total E2F1 protein and reduced levels of pRb protein compared to normal.

17. A PRMT5 inhibitor for use in treating a pRb defective cancer or for use in treating a cancer whose cells express reduced levels of pRb protein compared to normal.

18. The PRMT5 inhibitor for use according to any one of claim 13 to 17, selected from the group consisting of: an antibody, RNA interference molecule, antisense oligonucleotide or a small molecule compound.

19. The PRMT5 inhibitor for use according to claim 14, wherein the small molecule compound PRMT5 inhibitor is selected from: GSK3326595 (pemrametostat), PF-6939999, JNJ-64619178 (onametostat) and LLY-283.

20. The PRMT5 inhibitor for use according to any one of the claims 13-19, wherein the cancer is selected from the group consisting of: selected from the group consisting of: leukaemia, lymphoma, multiple myeloma, lung cancer, liver cancer, breast cancer, head and neck cancer, neuroblastoma, thyroid carcinoma, skin cancer (including melanoma), oral squamous cell carcinoma, urinary bladder cancer, Leydig cell tumour, biliary cancer, such as cholangiocarcinoma or bile duct cancer, brain cancer, pancreatic cancer, colon cancer, colorectal cancer and gynaecological cancers, including ovarian cancer, endometrial cancer, fallopian tube cancer, uterine cancer and cervical cancer, including epithelia cervix carcinoma. In suitable embodiments, the cancer is leukaemia and can be selected from the group consisting of acute lymphoblastic leukaemia, acute myelogenous leukaemia (also known as acute myeloid leukaemia or acute non-lymphocytic leukaemia), acute promyelocytic leukaemia, acute lymphocytic leukaemia, chronic myelogenous leukaemia (also known as chronic myeloid leukaemia, chronic myelocytic leukaemia or chronic granulocytic leukaemia), chronic lymphocytic leukaemia, monoblastic leukaemia and hairy cell leukaemia. In further preferred embodiments, the cancer is acute lymphoblastic leukaemia. In a suitable embodiment the cancer is lymphoma, which may be selected from the group consisting of: Hodgkin's lymphoma; non-Hodgkin lymphoma; Burkitt's lymphoma; and small lymphocytic lymphoma.

21. The PRMT5 inhibitor for use according to claim 17, wherein the cancer is selected from: breast cancer, esophageal cancer, bladder cancer, lung cancer, hematopoietic cancer, lymphoma, medulloblastoma, rectum adenocarcinoma, colon adenocarcinoma, gastric cancer, pancreatic cancer, liver cancer, adenoid cystic carcinoma, lung adenocarcinoma, head and neck squamous cell carcinoma, brain tumors, hepatocellular carcinoma, renal cell carcinoma, melanoma, oligodendroglioma, ovarian clear cell carcinoma, and ovarian serous.
